(19) United States
(12) Patent Application Publication
(10) Pub. No.: US 2026/0036569 A1
(43) Pub. Date: Feb. 5, 2026

(54) DEVICES, SYSTEMS, AND METHODS FOR PROCESSING SAMPLES

(71) Applicant: MOLARITI, INC., Cambridge, MA (US)

(72) Inventor: Ganeshkumar VARADARAJALU, Billerica, MA (US)

(21) Appl. No.: 19/186,828

(22) Filed: Apr. 23, 2025

Related U.S. Application Data
(63) Continuation of application No. PCT/US2023/077775, filed on Oct. 25, 2023.
(60) Provisional application No. 63/419,551, filed on Oct. 26, 2022.
(60) Provisional application No. 63/419,559, filed on Oct. 26, 2022.

(51) Int. Cl. G01N 33/487

(52) U.S. Cl. CPC G01N 33/48721

(57) ABSTRACT

The present disclosure provides a nanopore device for processing a fluid sample, comprising: a substrate; a top cavity, wherein the top cavity comprises a first dielectric; a bottom cavity, wherein the bottom cavity comprises a second dielectric; a first dielectric membrane supported by the substrate and separating the top cavity and the bottom cavity; a first set and a second set of electrodes located proximate to the first dielectric membrane, wherein the first set and the second set of electrodes are configured to apply a first electric field to generate a corona discharge thereby forming one or more nanopores in the first dielectric membrane.

### CROSS-REFERENCE TO RELATED APPLICATIONS

This application is a continuation of international application No. PCT/US23/77775, filed Oct. 25, 2023, which claims the benefit of U.S. Provisional Application No. 63/419,559, filed Oct. 26, 2022, and U.S. Provisional Application No. 63/419,551, filed Oct. 26, 2022, each of which is entirely incorporated herein by reference.

### BACKGROUND

Current biomolecule size separation instruments are generally based on gel electrophoresis, which may rely on the mobilities of biomolecules in a gel matrix under the influence of an electric field. There can be various technical problems associated with the gel electrophoresis technique, for example, gel electrophoresis may not be capable of analyzing very low or very high concentrations of biomolecules; the observed bands may have a diffuse spread even for biomolecules of the same size, which can lead to low resolution and poor size distribution profile; it can be difficult to achieve accurate quantification of the concentration; the use of fluorophores and single use gel cassettes may add to the cost of analysis; and the analysis can be slow. Furthermore, there may be environmental concerns associated with gel electrophoresis due to the use of plastics and/or toxic chemicals.

### SUMMARY

In an aspect, the present disclosure provides a nanopore device for processing a fluid sample, comprising: a substrate; a top cavity, wherein the top cavity comprises a first dielectric; a bottom cavity, wherein the bottom cavity comprises a second dielectric; a first dielectric membrane supported by the substrate and separating the top cavity and the bottom cavity; a first set and a second set of electrodes located proximate to the first dielectric membrane, wherein the first set and the second set of electrodes are configured to apply a first electric field to generate a corona discharge thereby forming one or more nanopores in the first dielectric membrane.

In some embodiments, the first and second sets of electrodes are configured to apply the first electric field before contacting the device with the fluid sample. In some embodiments, the first dielectric or the second dielectric comprises a gas, a water, an electrolyte fluid, or a solid. In some embodiments, the first dielectric or the second dielectric comprises the fluid sample. In some embodiments, the fluid sample comprises deoxyribonucleic acid (DNA), ribonucleic acid (RNA), proteins, lipids, or carbohydrates suspended in a gas, a water, an electrolyte fluid, or a porous solid. In some embodiments, the first and second sets of electrodes are configured to electrically couple to the first dielectric or the second dielectric. In some embodiments, a pair of the first and second sets of electrodes is configured to apply the first electric field that facilitates in the forming of the corona discharge to thereby form the one or more nanopores between the pair of the first and second sets of electrodes. In some embodiments, the device further comprises a first electric field generator, wherein the first electric field generator is configured to apply the first electric field sufficient to generate the corona discharge by applying a pulsed or continuous AC or DC voltage signal between the pair of the first and second sets electrodes. In some embodiments, the first electric field generator is configured to generate the electric field with a field strength of about 1 kilovolt/meter (kV/m), 10 kV/m, 100 kV/m, or greater. In some embodiments, the first electric field is configured to generate the corona discharge near an edge of the first set of electrodes or near an edge of the second set of electrodes. In some embodiments, the first electric field is configured to generate the corona discharge near an edge of a pair of the first set of electrodes, near an edge of a pair of the second set of electrodes, or a combination of both. In some embodiments, the edges of the pair of the first and second sets of electrodes are configured or formed substantially as a sharp convex tip. In some embodiments, the corona discharge is configured to increase a temperature of the device by at most about 10, 1, or 0.1 degrees Celsius or less. In some embodiments, the corona discharge is configured to increase a temperature near the first or second sets of electrodes by at most about 10, 1, or 0.1 degrees Celsius or less. In some embodiments, the corona discharge is configured to form the one or more nanopores with a diameter ranging from about 0.1 nanometers (nm) to about 10 micrometers (μm). In some embodiments, the substrate comprises a material of one or more combinations of silicon, glass, quartz, silicon on insulator (SOI), gallium arsenide, gallium nitride, silicon carbide, ceramics, aluminum nitride, aluminum oxide, silicon nitride, or silicon dioxide. In some embodiments, the first dielectric membrane comprises a material of one or more combinations of silicon nitride, silicon dioxide, graphene, boron nitride, tungsten disulfide, MXene, or molybdenum disulfide. In some embodiments, the first dielectric membrane is configured with a thickness ranging from about 0.1 nanometers (nm) to about 100 nm.

In some embodiments, the device further comprises a third set and a fourth set of electrodes configured to apply a second electric field, wherein the third set is located proximate to the first dielectric and the fourth set is located proximate to the second dielectric. In some embodiments, the third and fourth sets of electrodes are configured to apply the second electric field to generate an electrical are discharge to thereby form the one or more nanopores in the first dielectric membrane. In some embodiments, the combination of the corona discharge and the electrical are discharge is configured to form the one or more nanopores in the first dielectric membrane. In some embodiments, the third and fourth sets of electrodes are configured to generate the electrical are discharge through the first dielectric, the second dielectric, or the first dielectric membrane. In some embodiments, the third and fourth sets of electrodes are configured to apply the second electric field before contacting the device with the fluid sample or before generating the corona discharge. In some embodiments, the second electric field is configured to generate the electrical are discharge by exceeding a dielectric breakdown of the first dielectric, the second dielectric, or the first dielectric membrane. In some embodiments, the corona discharge is configured to concentrate the second electric field near the corona discharge to improve a location accuracy of the one or more nanopores by at least about 1%, 5%, 10%, or greater compared to without use of the corona discharge. In some embodiments, the third set of electrodes is configured to electrically couple to the first dielectric, and wherein the fourth set of electrodes is configured to electrically couple to the second dielectric.

In some embodiments, the pair of the third and fourth sets of electrodes is configured to apply the second electric field that facilitates in the forming of the electrical are discharge to thereby form the one or more nanopores in the first dielectric membrane. In some embodiments, the third and fourth sets of electrodes are configured to be integrated into the device, and wherein the electrodes are fabricated from a material comprising one or more combinations of gold, chromium, aluminum, platinum, iridium, or titanium. In some embodiments, the device further comprises a second electric field generator, wherein the second electric field generator is configured to apply the second electric field sufficient to generate the electrical are discharge by applying a pulsed or continuous AC or DC voltage signal between a pair of the third and fourth sets electrodes. In some embodiments, the second electric field generator is configured to generate the second electric field with a strength of about 1 kV/m, 10 kV/m, 100 kV/m, or greater. In some embodiments, the combination of the corona discharge and the electrical are discharge is configured to increase a temperature of the device by at most about 1000, 100, 10, 1, or 0.1 degrees Celsius or less. In some embodiments, the combination of the corona discharge and the electrical are discharge is configured to increase a temperature near the first, second, third, or fourth sets of electrodes by at most about 1000, 100, 10, 1, or 0.1 degrees Celsius or less. In some embodiments, the combination of the corona discharge and the electrical are discharge is configured to form the one or more nanopores with a diameter ranging from about 0.1 nm to about 10 μμm.

In some embodiments, the device further comprises one or more sensors configured to measure a tunnel current between a pair of the first and second sets of electrodes or an ionic current between a pair of the third and fourth set of electrodes. In some embodiments, the pair of the first and second sets of electrodes is configured to measure the tunnel current. In some embodiments, the pair of the third and fourth sets of electrodes is configured to measure the ionic current. In some embodiments, the one or more sensors are configured to be integrated into the device.

In some embodiments, the device further comprises a second dielectric membrane, wherein the second dielectric membrane forms a layer between the first dielectric membrane and the substrate or the second dielectric. In some embodiments, the second dielectric membrane is configured to support the first dielectric membrane during the forming of the one or more nanopores and to be removed by a chemical etching process after the forming of the one or more nanopores.

In some embodiments, the one or more nanopores comprises at least about 1, 10, 100, 1000, 10000, 100000, 1000000, or more nanopores. In some embodiments, the device is configured to electrically couple the one or more nanopores thereby forming one or more nanopore cells. In some embodiments, the device is configured to electrically couple the one or more nanopore cells thereby forming one or more nanopore arrays. In some embodiments, the one or more nanopore arrays are configured to perform high-throughput processing of the fluid sample. In some embodiments, the high-throughput processing comprises sequencing DNA at a rate of at least about 1, 10, 100, 1000, 10000, or greater kilobases/s (kb/s). In some embodiments, the device is configured to reform the one or more nanopores in the first membrane. In some embodiments, the device further comprises a top layer coupled to the first dielectric and a bottom layer coupled to the second dielectric, wherein the top and bottom layers are configured to contain or seal the fluid sample or guide a flow of the fluid sample through the device. In some embodiments, the device further comprises a fifth electrode and a sixth electrode configured to generate a dielectrophoretic (DEP) force with the first and second sets of electrodes. In some embodiments, the DEP force is used to concentrate biomolecules contained in the fluid sample.

In another aspect, the present disclosure provides a method of manufacturing a nanopore device for processing a fluid sample, the method comprising: (a) providing a device, the device comprising: a substrate; a top cavity; a bottom cavity; a first dielectric membrane; a first set and a second set of electrodes; and a third set and a fourth set of electrodes; (b) applying a first electric field between the first and second sets of electrodes to generate a corona discharge near an edge of the first and second sets of electrodes; and (c) applying a second electric field between the third and fourth sets of electrodes to generate an electrical are discharge through the corona discharge, wherein a combination of the corona discharge and the electrical are discharge form one or more nanopores in the first dielectric membrane.

In some embodiments, (a) comprises: (i) depositing the first dielectric membrane on the substrate; (ii) forming the bottom cavity in the substrate underneath the first dielectric membrane, wherein the bottom cavity comprises a dielectric; (iii) depositing the first set and the second set of electrodes on top of the first dielectric membrane; (iv) forming the top cavity on top of the first dielectric membrane and on top of the first and second sets of electrodes, wherein the top cavity comprises an additional dielectric; (v) depositing the third set of electrodes on a top side of the top cavity; and (vi) depositing the fourth set of electrodes on a bottom side of the bottom cavity.

In some embodiments, the depositing in (iii) comprises depositing the first and second sets of electrodes proximate to the dielectric, the additional dielectric, or the first dielectric membrane. In some embodiments, the first and second sets of electrodes comprise a material of one or more combinations of gold, chromium, aluminum, platinum, iridium, or titanium.

In some embodiments, the depositing in (vi) comprises depositing the third set of electrodes proximate to the first dielectric and depositing the fourth set of electrodes proximate to the second dielectric. In some embodiments, the third and fourth sets of electrodes comprise a material of one or more combinations of gold, chromium, aluminum, platinum, iridium, or titanium.

In some embodiments, the method further comprises generating the first electric field by applying a pulsed or continuous AC or DC voltage signal between the first and second sets electrodes. In some embodiments, the first electric field comprises a level of at least about 1 kV/m, 10 kV/m, 100 kV/m, or greater. In some embodiments, the method further comprises generating the second electric field by applying a pulsed or continuous AC or DC voltage signal between the third and fourth sets electrodes. In some embodiments, the second electric field comprises a level of at least about 1 kV/m, 10 kV/m, 100 kV/m, or greater. In some embodiments, the device further comprises a second dielectric membrane. In some embodiments, (a) further comprises depositing the second dielectric membrane between the first dielectric membrane and the substrate or the second dielectric. In some embodiments, the device further comprises a top layer and a bottom layer. In some embodiments, (a) further comprises forming the top layer to a top of the first dielectric and forming the bottom layer to a bottom of the second dielectric. In some embodiments, the top and bottom layers are configured to contain or seal the fluid sample or to guide a flow of the fluid sample through the device.

In some embodiments, (a) further comprises: (1) generating a plurality of the nanopore devices; (2) electrically connecting at least one nanopore device of the plurality of nanopore devices to at least one other nanopore device of the plurality of nanopore devices to form a plurality of nanopore cells; and (3) electrically connecting at least one nanopore cell of the plurality of nanopore cells to at least one other nanopore cell of the plurality of nanopore cells to form a plurality of nanopore arrays.

In some embodiments, the plurality of nanopore arrays is configured to perform high-throughput processing of the fluid sample. In some embodiments, the device further comprises a fifth electrode and a sixth electrode configured to generate a dielectrophoretic (DEP) force with the first and second sets of electrodes.

In another aspect, the present disclosure provide a method for processing a fluid sample using one or more nanopores, the method comprising: (a) providing a device, the device comprising: a substrate; a top cavity; a bottom cavity; a first dielectric membrane; a first set and a second set of electrodes configured to generate a corona discharge; and a third set and a fourth set of electrodes configured to generate an electrical are discharge, wherein the one or more nanopores are formed in the first dielectric membrane using a combination of the corona discharge and the electrical are discharge; (b) flowing the fluid sample through the one or more nanopores; and (c) processing the fluid sample as the fluid sample flows through the one or more nanopores.

In some embodiments, the method further comprises using the first, second, third, or fourth sets of electrodes to form the one or more nanopores. In some embodiments, the first and second sets of electrodes are further configured to process one or more events associated with the processing of the fluid sample. In some embodiments, the fluid sample comprises deoxyribonucleic acid (DNA), ribonucleic acid (RNA), proteins, lipids, or carbohydrates suspended in a gas, a water, an electrolyte fluid, or a porous solid. In some embodiments, the one or more events comprises events associated with analyzing biomolecules, measuring biomolecules, purifying biomolecules, concentrating biomolecules, or sequencing biomolecules, In some embodiments, the first and second sets of electrodes are further configured to measure a tunnel current. In some embodiments, the third and fourth sets of electrodes are further configured to measure an ionic current. In some embodiments, the device further comprises a fifth electrode and a sixth electrode configured to generate a dielectrophoretic (DEP) force with the first and second sets of electrodes. In some embodiments, the method further comprises using the DEP force to concentrate biomolecules contained in the fluid sample. In some embodiments, (b) comprises flowing the fluid sample through the one or more nanopores via capillary, pressure driven, electro osmotic flow, or electrowetting mechanism.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

Where reference is made to a URL or other such identifier or address, such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A depicts an example nanopore unit cell, according to some embodiments;

FIG. 1B depicts another example nanopore unit cell, according to some embodiments;

FIG. 2 shows a top view schematic of an example nanopore chip with a plurality of nanopore unit cell (e.g., nanopore unit cell block), according to some embodiments;

FIG. 3 shows an example nanopore array, according to some embodiments;

FIG. 4 shows an example system for sample processing or biomolecule analysis, according to some embodiments;

FIGS. 5A and 5B show example setups for generating corona discharge, according to some embodiments;

FIG. 6 shows an example setup for forming a nanopore, according to some embodiments;

FIGS. 7A-7D show example setups for forming a nanopore, according to some embodiments;

FIG. 8 shows an example setup for corona discharge-assisted controlled dielectric breakdown nanopore creation, according to some embodiments;

FIG. 9 shows another example setup for corona discharge-assisted controlled dielectric breakdown nanopore creation, according to some embodiments;

FIG. 10 shows an example setup for are discharge nanopore creation, according to some embodiments;

FIG. 11 shows another example setup for are discharge nanopore creation, according to some embodiments;

FIG. 12 shows an example size distribution chart, according to some embodiments;

FIG. 13 depicts a biomolecule translocating from a top chamber to a bottom chamber of a nanopore unit cell, according to some embodiments;

FIG. 14 shows an example measurement of current vs time in the nanopore, according to some embodiments;

FIG. 15 shows a computer system, according to some embodiments; and

FIG. 16 shows an example nanopore device for sample processing or biomolecule analysis.

### DETAILED DESCRIPTION

While various embodiments of the present disclosure have been shown and described herein, such embodiments are provided by way of example only. Numerous variations, changes, and substitutions may occur without departing from the present disclosure. It can be understood that various alternatives to the embodiments of the present disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used in the specification and claims, the singular forms “a,” “an,” and “the” can include plural references unless the context clearly dictates otherwise. For example, the term “a biomolecule” can include a plurality of biomolecules.

The terms “about,” and “approximately,” as used interchangeably herein, generally refer to within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, such as within 5-fold or within 2-fold of a value. Where particular values are described, unless otherwise stated, the term “about” can mean within an acceptable error range for the particular value.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the properties sought to be obtained by the present disclosure.

As used herein. “comprising” is generally open ended and means the elements recited, or their equivalent in structure or function, plus any other element or elements which are not recited. The terms “having” and “including” are also to be construed as open ended unless the context suggests otherwise.

As used herein, “optional” or “optionally” generally means that the subsequently described event or circumstance does or does not occur and that the description includes instances where said event or circumstance occurs and instances where it does not. For example, an optionally variant portion means that the portion is variant or non-variant.

The terms “polynucleotide.” and “nucleic acid.” as used interchangeably herein, generally refer to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be deoxyribonucleic acid (DNA). A polynucleotide can be ribonucleic acid (RNA). A polynucleotide can have any three-dimensional structure, and can perform any function, known or unknown. A polynucleotide can comprise one or more analogs (e.g., backbone, sugar, or nucleobase analogs). Non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs. CpG islands, methyl-7-guanosine, methylated nucleotides, inosine, thiouridine, pseudourdine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, complementary DNA (cDNA, such as double-strand cDNA (dd-cDNA) or single-stranded cDNA (ss-cDNA)), circulating tumor DNA (ctDNA), damaged DNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes (e.g., fluorescence in situ hybridization (FISH) probes), and primers.

The term “sequencing,” as used herein, generally refers to a procedure for determining the order in which nucleotides occur in a target nucleotide sequence. Methods of sequencing can comprise high-throughput sequencing, such as, for example, next-generation sequencing (NGS). Sequencing may be whole-genome sequencing or targeted sequencing. Sequencing may be single molecule sequencing or massively parallel sequencing. Next-generation sequencing methods can be useful in obtaining millions of sequences in a single run. In some cases, sequencing may be performed using one or more nanopore sequencing methods, e.g., sequencing-by-synthesis, sequencing-by-ligation, or sequencing-by-cleavage.

The term “nanopore,” as used herein, generally refers to a pore, channel, or passage formed or otherwise provided in a membrane. A nanopore can be a biological nanopore, solid state nanopore, hybrid biological-solid state nanopore, a variation thereof, or a combination thereof. The membrane may be an organic membrane, e.g., a lipid bilayer, or a synthetic membrane, e.g., a membrane formed of a polymeric material such as a protein nanopore. The membrane may be a solid state membrane (e.g., silicon substrate). The nanopore may be disposed adjacent or in proximity to a sensing circuit or an electrode coupled to a sensing circuit, such as, for example, a complementary metal-oxide semiconductor (CMOS) or field effect transistor (FET) circuit. The nanopore may be part of the sensing circuit.

As used herein, the term “nanopore site” generally refers to a location where a nanopore can be created, where a nanopore is located, or where a nanopore was previously located.

Solid state nanopores can be fabricated by at least one of the methods: 1) transmission electron microscopy (TEM); 2) controlled dielectric breakdown (CDB); 3) laser etching (LE); 4) laser assisted controlled dielectric breakdown (LCDB); and 5) electron beam lithography (EBL). Technical problems may be associated with these methods. For example, these methods can require expensive instruments or complicated alignment. In addition, these methods may produce only a single nanopore. Hence, there remains an unmet need for scalable fabrication processes to build devices (e.g., nanopore devices) with an array of nanopores capable of high throughput biomolecule analysis.

The present disclosure provides devices, systems, and methods for processing a fluid sample. The present disclosure further provides methods for manufacturing or fabricating a nanopore device for processing a fluid sample. The scalable manufacturing or fabrication process provided herein, e.g., massively parallel nanopore fabrication processes with corona discharge and/or are discharge, can create nanopores at a massive (e.g., millions or more) scale, useful for high throughput biomolecule analysis.

In some embodiments, a device, system, and method provided herein can be used for analyzing biomolecules, e.g., nucleic acids, polynucleotides, and polypeptides. In some embodiments, a device, system, and method disclosed herein can be used for biomolecule characterization, analysis, and/or sequencing. In some embodiments, a device, system, and method described herein can be used to determine or measure multiple characteristics of the biomolecules. In some embodiments, a biomolecule can comprise deoxyribonucleic acid (DNA), ribonucleic acid (RNA), proteins, lipids, or carbohydrates. In some embodiments, the characteristics can comprise sizing and/or concentrating of biomolecules. In some embodiments, the characteristics can comprise sequencing of a biomolecule (e.g., DNA, RNA, or proteins).

### Devices for Processing Samples

In some embodiments, the present disclosure provides a device for processing a fluid sample. In some embodiments, the device can comprise a nanopore device. In some embodiments, the nanopore device can comprise a plurality of nanopore unit cells, nanopore chips, and/or nanopore chip arrays.

In some embodiments, the nanopore device can comprise a substrate; a top cavity comprising a first dielectric; a bottom cavity comprising a second dielectric; a first dielectric membrane that is supported by the substrate and configured to separate the top cavity and the bottom cavity; a first set and a second set of electrodes located proximate to the first dielectric membrane, wherein the first set and the second set of electrodes are configured to apply a first electric field to generate a corona discharge thereby forming one or more nanopores in the first dielectric membrane.

In some embodiments, the first and second sets of electrodes can be configured to apply the first electric field before contacting the nanopore device with the fluid sample.

In some embodiments, the nanopore device can comprise a second dielectric membrane between the first dielectric membrane and the substrate. In some embodiments, the second dielectric membrane can comprise a same composition as the first dielectric membrane. In some embodiments, the second dielectric membrane can comprise a different composition from the first dielectric membrane. In some embodiments, the second dielectric membrane can comprise silicon dioxide. In some embodiments, the second dielectric membrane can be configured to support the first dielectric membrane during the forming of the one or more nanopores. In some embodiments, the second dielectric membrane can have a thickness from about 20 nm to about 10 μm. In some embodiments, the second dielectric membrane can stay in the nanopore device. In some embodiments, at least a portion of the second dielectric membrane can be removed, e.g., by a chemical etching process after the forming of the one or more nanopores. In some embodiments, a portion of the second dielectric membrane under the nanopore site can be removed. In some embodiments, after the nanopore formation, the second dielectric membrane may only contact the substrate but not contact the second dielectric. In some embodiments, after the nanopore formation, the portion of second dielectric membrane that does not contact the substrate may be removed.

In some embodiments, the nanopore device can comprise additional external electrodes or embedded electrodes.

In some embodiments, the substrate can comprise silicon, glass, quartz, silicon on insulator (SOI), gallium arsenide, gallium nitride, silicon carbide, ceramics, aluminum nitride, aluminum oxide, silicon nitride, silicon dioxide, or a combination thereof. In some embodiments, the substrate can comprise a silicon wafer. In some embodiments, the substrate can comprise a crystalline silicon wafer. In some embodiments, the silicon wafer can comprise a given doping concentration. In some embodiments, the substrate, e.g., silicon wafer, can comprise dopants, such as phosphorus, arsenic, antimony, or boron. In some embodiments, the doped silicon wafer can have a resistivity from about 0.001 ohm·cm to about 100 ohm·cm. In some embodiments, the silicon wafer can be undoped. In some embodiments, the undoped silicon wafer can have a resistivity greater than 20000 ohm·cm. In some embodiments, the substrate can comprise a glass. In some embodiments, the substrate can comprise doped glass. In some embodiments, the substrate can comprise undoped glass. In some embodiments, undoped silicon wafer or undoped glass wafer may reduce noise level in measuring a signal (e.g., tunneling signal). In some embodiments, undoped silicon wafer or undoped glass wafer may increase a signal to noise ratio in such measurements.

In some embodiments, the first dielectric can comprise a fluid, such as a gas or a liquid (e.g., water) or combination thereof, or a solid (porous or non-porous solid). In some embodiments, the first dielectric can comprise air, nitrogen, oxygen, argon, tetrafluoromethane (CF₄), nitrogen trifluoride (NF₃), trifluoromethane (CHF₃), or sulfur hexafluoride (SF₆). In some embodiments, the first dielectric can comprise porcelain, glass, agarose gel, hydrogel, or plastics. In some embodiments, the first dielectric can comprise an electrolyte fluid. In some embodiments, the first dielectric can comprise a solution, e.g., KCl or LiCl solutions. In some embodiments, the first dielectric can comprise electrolyte solutions (buffers) such as phosphate-buffered saline (PBS), (4-(2-Hydroxyethyl)piperazine-1-ethane-sulfonic acid) buffer (HEPES). Tris-EDTA with low conductivity, or water of a specific conductivity or resistivity. In some embodiments, the water can have a resistivity from about 10 kilo-ohm meter (kΩ·m) to about 200 kΩ·m. In some embodiments, the water can have a resistivity from about 180 kΩ·m.

In some embodiments, the second dielectric can comprise a fluid, such as a gas or a liquid (e.g., water) or combination thereof, or a solid (porous or non-porous solid). In some embodiments, the second dielectric can comprise air, nitrogen, oxygen, argon, tetrafluoromethane (CF₄), nitrogen trifluoride (NF₃), trifluoromethane (CHF₃), or sulfur hexafluoride (SF₆). In some embodiments, the second dielectric can comprise porcelain, glass, agarose gel, hydrogel, or plastics. In some embodiments, the second dielectric can comprise an electrolyte fluid. In some embodiments, the second dielectric can comprise a solution, e.g., KCl or LiCl solutions. In some embodiments, the second dielectric can comprise electrolyte solutions (buffers) such as phosphate-buffered saline (PBS), (4-(2-Hydroxyethyl)piperazine-1-ethane-sulfonic acid) buffer (HEPES). Tris-EDTA with low conductivity, or water of a specific conductivity or resistivity. In some embodiments, the water can have a resistivity from about 10 kilo-ohm meter (kΩ·m) to about 200 kΩ·m. In some embodiments, the water can have a resistivity from about 180 kΩ·m.

In some embodiments, the first dielectric and/or the second dielectric can comprise the fluid sample. In some embodiments, the fluid sample can comprise deoxyribonucleic acid (DNA), ribonucleic acid (RNA), proteins, lipids, or carbohydrates suspended in a gas, a water, an electrolyte fluid, or a porous solid (e.g., gel). In some embodiments, the porous solid can slow down the translocation of biomolecules, e.g., DNA, through the nanopore.

In some embodiments, the first dielectric membrane can comprise any suitable dielectric material. In some embodiments, the first dielectric membrane can comprise a 2-dimensional material. In some embodiments, the first dielectric membrane can comprise silicon nitride (Si₃N₄), silicon dioxide (SiO₂), graphene, boron nitride (e.g., hexagonal boron nitride), tungsten disulfide, MXene, molybdenum disulfide (MoS₂), silicon carbide (SiC), titanium dioxide (TiO₂), zirconium dioxide (ZrO₂), aluminum oxide (Al₂O₃), polytetrafluoroethylene (PTFE), polyacrylonitrile (PAN), high density polyethylene (HDPE), or a combination thereof. In some embodiments, the dielectric membrane can have a thickness from about 1 angstrom (Å) to about 5 Å, from about 1 Å to about 1 nanometer (nm), from about 1 Å to about 5 nm, from about 1 Å to about 10 nm, from about 1 Å to about 50 nm, from about 1 Å to about 100 nm, from about 5 Å to about 1 nm, from about 5 Å to about 5 nm, from about 5 Å to about 10 nm, from about 5 Å to about 50 nm, from about 5 Å about 100 nm, from about 1 nm to about 5 nm, from about 1 nm to about 10 nm, from about 1 nm to about 50 nm, from about 1 nm to about 100 nm, from about 5 nm to about 10 nm, from about 5 nm to about 50 nm, from about 5 nm to about 100 nm, from about 10 nm to about 50 nm, from about 10 nm to about 100 nm, or from about 50 nm to about 100 nm.

In some embodiments, the first and second sets of electrodes can be configured to electrically couple to the first dielectric or the second dielectric. In some embodiments, a pair of the first and second sets of electrodes can be configured to apply the first electric field that facilitates the forming of the corona discharge to thereby form the one or more nanopores between the pair of the first and second sets of electrodes. In some embodiments, the electrodes can comprise platinum, gold, chromium, aluminum, titanium, tungsten, molybdenum, niobium, hafnium, iridium, osmium, zirconium, palladium, iron, or a combination thereof. In some embodiments, the electrodes can comprise platinum. In some embodiments, at least one electrode of the pair of the first and second sets of electrodes can comprise a pointed edge or be configured or formed substantially as a sharp convex tip.

In some embodiments, the nanopore device can comprise a first electric field generator, wherein the first electric field generator can be configured to apply the first electric field sufficient to generate the corona discharge by applying a pulsed or continuous alternating current (AC) or direct current (DC) voltage signal between the pair of the first and second sets electrodes. In some embodiments, the first electric field generator can be configured to generate the electric field with a field strength of at least about 1 kilovolt/meter (kV/m), at least about 5kV/m, at least about 10 kV/m, at least about 20 kV/m, at least about 30 kV/m, at least about 40) kV/m, at least about 50) kV/m, at least about 60 kV/m, at least about 70 kV/m, at least about 80) kV/m, at least about 90) kV/m, at least about 100 kV/m, at least about 1 megavolt/meter (MV/m), at least about 10 MV/m, at least about 100 MV/m, or greater.

In some embodiments, the first electric field can be configured to generate the corona discharge near an edge of the first set of electrodes or near an edge of the second set of electrodes. In some embodiments, the first electric field can be configured to generate the corona discharge near an edge of a pair of the first set of electrodes, near an edge of a pair of the second set of electrodes, or a combination of both. In some embodiments, the edges of the pair of the first and second sets of electrodes can be configured or formed substantially as a sharp convex tip.

In some embodiments, the corona discharge can be a positive corona discharge (e.g., near an edge of a positive electrode). In some embodiments, the corona discharge can be a negative corona discharge (e.g., near an edge of a negative electrode).

In some embodiments, the corona discharge can increase a temperature of the nanopore device by at least about 0.1° C., at least about 0.5° C., at least about 1° C., at least about 5° C., at least about 10° C., or more. In some embodiments, the corona discharge can increase a temperature of the nanopore device by at most about 10 degrees Celsius (° C.), at most about 5° C., at most about 1° C., at most about 0.5° C., at most about 0.1° C., or less.

In some embodiments, the corona discharge can increase a temperature near the first or second sets of electrodes by at least about 0.1° C., at least about 0.5° C., at least about 1° C., at least about 5° C., at least about 10° C., or more. In some embodiments, the corona discharge can increase a temperature near the first or second sets of electrodes by at most about 10 degrees Celsius (° C.), at most about 5° C., at most about 1° C., at most about 0.5° C., at most about 0.1° C., or less.

In some embodiments, the corona discharge can be configured to form the one or more nanopores with a diameter from about 0.1 nanometers (nm) to about 1 nm, from about 0.1 nm to about 5 nm, from about 0.1 nm to about 10 nm, from about 0.1 nm to about 50 nm, from about 0.1 nm to about 100 nm, from about 0.1 nm to about 500 nm, from about 0.1 nm to about 1 micrometer (μm), from about 0.1 nm to about 10 μm, from about 1 nm to about 5 nm, from about 1 nm to about 10 nm, from about 1 nm to about 50 nm, from about 1 nm to about 100 nm, from about 1 nm to about 500 nm, from about 1 nm to about 1 μm, from about 1 nm to about 10 μm, from about 5 nm to about 10 nm, from about 5 nm to about 50 nm, from about 5 nm to about 100 nm, from about 5 nm to about 500 nm, from about 5 nm to about 1 μm, from about 5 nm to about 10 μm, from about 10 nm to about 50 nm, from about 10 nm to about 100 nm, from about 10 nm to about 500 nm, from about 10 nm to about 1 μm, from about 10 nm to about 10 μm, from about 50 nm to about 100 nm, from about 50 nm to about 500 nm, from about 50 nm to about 1 μm, from about 50 nm to about 10 μm, from about 100 nm to about 500 nm, from about 100 nm to about 1 μm, from about 100 nm to about 10 μm, from about 500 nm to about 1 μm, from about 500 nm to about 10 μm, or from about 1 μm to about 10 μm.

In some embodiments, the nanopore device can comprise a third set and a fourth set of electrodes configured to apply a second electric field. In some embodiments, the third set of electrodes can be located proximate to the first dielectric (e.g., in the top cavity) and the fourth set of electrodes can be located proximate to the second dielectric (e.g., in the bottom cavity). In some embodiments, the third set of electrodes can be configured to electrically couple to the first dielectric. In some embodiments, the fourth set of electrodes can be configured to electrically couple to the second dielectric. In some embodiments, the third and fourth sets of electrodes can be configured to apply the second electric field to generate an electrical are discharge (or are discharge, used interchangeably herein) to thereby form the one or more nanopores in the first dielectric membrane.

In some embodiments, the third and fourth sets of electrodes can provide additional electric strength to form the one or more nanopores in the first dielectric membrane. In some embodiments, the combination of the corona discharge and the electrical are discharge can be configured to form the one or more nanopores in the first dielectric membrane.

In some embodiments, the third and fourth sets of electrodes can be configured to generate the electrical are discharge through the first dielectric, the second dielectric, and/or the first dielectric membrane. In some embodiments, the third and fourth sets of electrodes can be configured to generate the electrical are discharge through the first dielectric, the second dielectric, and the first dielectric membrane.

In some embodiments, the combination of the corona discharge and the electrical are discharge can increase a temperature of the device by at least about 0.1° C., at least about 0.5° C., at least about 1° C., at least about 5° C., at least about 10° C., at least about 100° C., at least about 500° C., at least about 1000° C., at least about 10000° C., at least about 50000° C., or more. In some embodiments, the combination of the corona discharge and the electrical are discharge can increase a temperature of the nanopore device by at most about 50000° C., at most about 10000° C., at most about 1000° C., at most about 100° C., at most about 10° C., at most about 5° C., at most about 1° C., at most about 0.5° C., at most about 0.1° C., or less.

In some embodiments, the combination of the corona discharge and the electrical are discharge can increase a temperature near the first, second, third, or fourth sets of electrodes by at least about 0.1° C., at least about 0.5° C., at least about 1° C., at least about 5° C., at least about 10° C., at least about 100° C., at least about 500° C., at least about 1000° C., at least about 10000° C., at least about 50000° C., or more. In some embodiments, the combination of the corona discharge and the electrical are discharge can increase a temperature near the first, second, third, or fourth sets of electrodes by at most about 50000® C., at most about 10000° C., at most about 1000° C., at most about 100° C., at most about 10° C., at most about 5° C., at most about 1° C., at most about 0.5° C., at most about 0.1° C., or less.

In some embodiments, the combination of the corona discharge and the electrical are discharge can be configured to form the one or more nanopores with a diameter from about 0.1 nanometers (nm) to about 1 nm, from about 0.1 nm to about 5 nm, from about 0.1 nm to about 10 nm, from about 0.1 nm to about 50 nm, from about 0.1 nm to about 100 nm, from about 0.1 nm to about 500 nm, from about 0.1 nm to about 1 micrometer (μm), from about 0.1 nm to about 10 μm, from about 1 nm to about 5 nm, from about 1 nm to about 10 nm, from about 1 nm to about 50 nm, from about 1 nm to about 100 nm, from about 1 nm to about 500 nm, from about 1 nm to about 1 μm, from about 1 nm to about 10 μm, from about 5 nm to about 10 nm, from about 5 nm to about 50 nm, from about 5 nm to about 100 nm, from about 5 nm to about 500 nm, from about 5 nm to about 1 μm, from about 5 nm to about 10 μm, from about 10 nm to about 50 nm. from about 10 nm to about 100 nm, from about 10 nm to about 500 nm, from about 10 nm to about 1 μm, from about 10 nm to about 10 μm, from about 50 nm to about 100 nm, from about 50 nm to about 500 nm, from about 50 nm to about 1 μm, from about 50 nm to about 10 μm, from about 100 nm to about 500 nm, from about 100 nm to about 1 μm, from about 100 nm to about 10 μm, from about 500 nm to about 1 μm, from about 500 nm to about 10 μm, or from about 1 μm to about 10 μm.

In some embodiments, the third and fourth sets of electrodes can be configured to apply the second electric field before contacting the nanopore device with the fluid sample or before generating the corona discharge. In some embodiments, the third and fourth sets of electrodes can be configured to apply the second electric field after contacting the nanopore device with the fluid sample or after generating the corona discharge. In some embodiments, the third and fourth sets of electrodes can be configured to apply the second electric field simultaneously at the same time as generating the corona discharge.

In some embodiments, the second electric field can be configured to generate the electrical are discharge by exceeding a dielectric breakdown of the first dielectric, the second dielectric, or the first dielectric membrane. In some embodiments, the corona discharge can be configured to concentrate the second electric field near the corona discharge to improve a location accuracy of the one or more nanopores by at least about 1%, at least about 5%, at least about 10%, or greater, compared to without use of the corona discharge.

In some embodiments, the third and fourth sets of electrodes can comprise a pair of electrodes configured to apply the second electric field that facilitates in the forming of the electrical are discharge to thereby form the one or more nanopores in the first dielectric membrane.

In some embodiments, the third and fourth sets of electrodes can be external to the nanopore device. In some embodiments, the third and fourth sets of electrodes can be integrated into the device (e.g., embedded into the device). In some embodiments, the third and fourth sets of electrodes can be fabricated from a material comprising one or more combinations of gold, chromium, aluminum, platinum, iridium, or titanium.

In some embodiments, the nanopore device can comprise a second electric field generator. In some embodiments, the second electric field generator can be configured to apply the second electric field sufficient to generate the electrical are discharge by applying a pulsed or continuous AC or DC voltage signal between a pair of the third and fourth sets electrodes.

In some embodiments, the second electric field generator can generate the second electric field with a strength of at least about 1 kV/m, at least about 5 kV/m, at least about 10 kV/m, at least about 50 kV/m, at least about 100 kV/m, at least about 1 MV/m, at least about 10 MV/m, at least about 100 MV/m, at least about 1000 MV/m, at least about 5000 MV/m, or greater.

In some embodiments, the nanopore device can further comprise a fifth electrode and a sixth electrode. In some embodiments, the nanopore device can be configured to generate a dielectrophoretic (DEP) force, e.g., by applying an AC electric field (or a third electric field). In some embodiments, the first set of electrodes, the second set of electrodes, the fifth electrode, and the sixth electrode can be configured to generate the DEP force, e.g., by applying an AC electric field. In some embodiments, a pair of the first and second sets of electrodes, the fifth electrode, and the sixth electrode can be configured to generate the DEP force, e.g., by applying an AC electric field. In some embodiments, the DEP force can be used to concentrate biomolecules contained in the fluid sample after the fluid sample comprising the biomolecules is delivered to the nanopore device.

In some embodiments, the nanopore device can comprise a third electric field generator, wherein the third electric field generator can be configured to apply the third electric field by applying a pulsed or continuous alternating current (AC) or direct current (DC) voltage signal between the pair of the fifth and sixth electrodes. In some embodiments, the third electric field generator can be configured to generate the electric field with a field strength of at least about 1 kV/m, at least about 5 kV/m, at least about 10 kV/m, at least about 20 kV/m, at least about 30 kV/m, at least about 40 kV/m, at least about 50 kV/m, at least about 60 kV/m, at least about 70 kV/m, at least about 80 kV/m, at least about 90 kV/m, at least about 100 kV/m, at least about 1 MV/m, at least about 10 MV/m, at least about 100 MV/m, or greater.

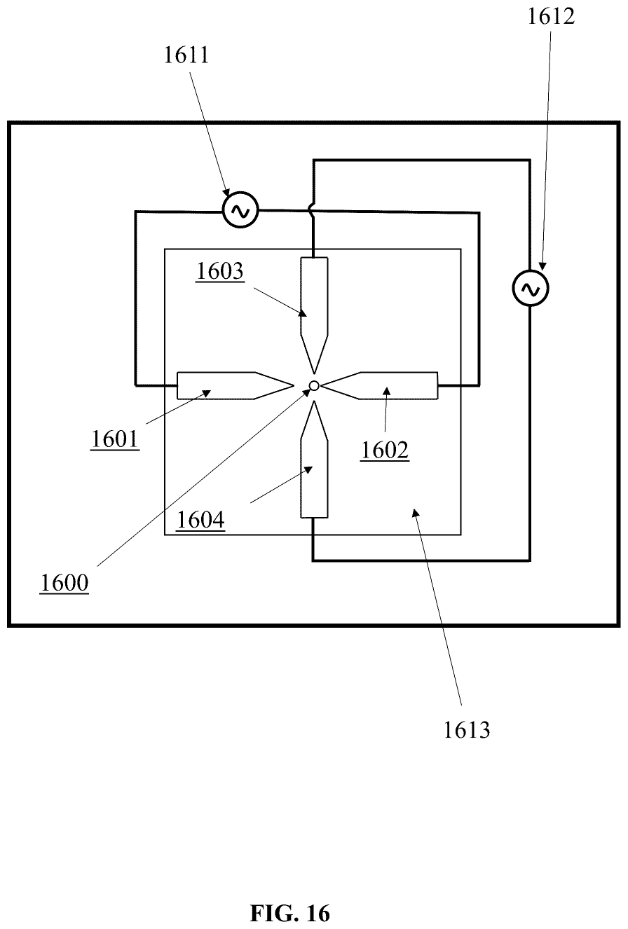

FIG. 16 shows an example nanopore device for sample processing or biomolecule analysis. The nanopore device can comprise a pair of electrodes 1601 and 1602 disposed proximate to the nanopore site 1600 and configured to generate corona discharge during nanopore formation. In the sample processing or biomolecule analysis (in fluid 1613), the pair of electrodes 1601 and 1602 can be configured to detect tunneling current (or tunnel current, as used interchangeably herein). An electric field generator 1611 (e.g., AC source) can generate an electric field between electrodes 1601 and 1602. The nanopore device can comprise another pair of electrodes 1603 and 1604 disposed proximate to the nanopore site 1600. The electrodes 1601, 1602, 1603, and 1604 can be configured to generate a DEP force. An additional electric field generator 1612 (e.g., AC source) can generate an additional electric field between electrodes 1603 and 1604.

In some embodiments, the nanopore device can further comprise one or more sensors configured to measure a tunnel current between a pair of the first and second sets of electrodes or an ionic current between a pair of the third and fourth set of electrodes. In some embodiments, the one or more sensors can be integrated into the nanopore device. In some embodiments, the one or more sensors can be operatively coupled to any one of the electrodes.

In some embodiments, the pair of the first and second sets of electrodes can be configured to measure the tunnel current. In some embodiments, the pair of the third and fourth sets of electrodes can be configured to measure the ionic current.

In some embodiments, the nanopore device can comprise at least about 1, at least about 10, at least about 100, at least about 1000, at least about 10000, at least about 100000, at least about 1000000, or more nanopores.

In some embodiments, the nanopore device can be configured to electrically couple the one or more nanopores thereby forming one or more nanopore cells or nanopore unit cells.

In some embodiments, the nanopore device can be configured to electrically couple the one or more nanopore unit cells thereby forming one or more nanopore arrays or nanopore blocks.

In some embodiments, the one or more nanopore arrays can be configured to perform high-throughput processing of the fluid sample or analyzing of biomolecules. In some embodiments, the high-throughput processing or analysis can comprise sequencing DNA at a rate of at least about 1, at least about 10, at least about 100, at least about 1000, at least about 10000, or greater kilobases/s (kb/s). In some embodiments, the high-throughput processing or analysis can comprise simultaneously processing or analyzing at least about 10, at least about 100, at least about 1000, at least about 10000, at least about 100000, or more samples.

In some embodiments, a nanopore may be damaged or clogged after usage. In some embodiments, the nanopore device provided herein can be configured to reform or regenerate the one or more nanopores in the first dielectric membrane. In some embodiments, the reforming or regeneration of the one or more nanopores in the first dielectric membrane can be achieved by any of the manufacturing/fabrication/forming method provided herein.

In some embodiments, the nanopore device can comprise a top layer coupled to the first dielectric and a bottom layer coupled to the second dielectric. The top and bottom layers can contain or seal the fluid sample or guide a flow of the fluid sample through the nanopore device.

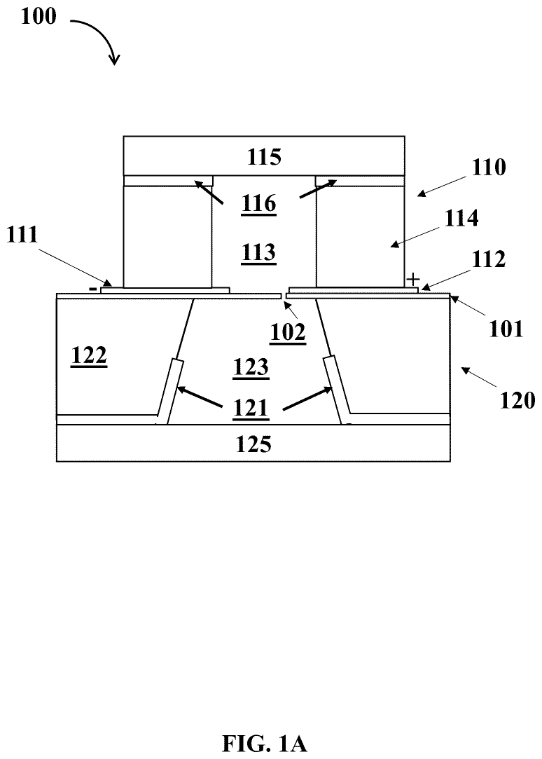

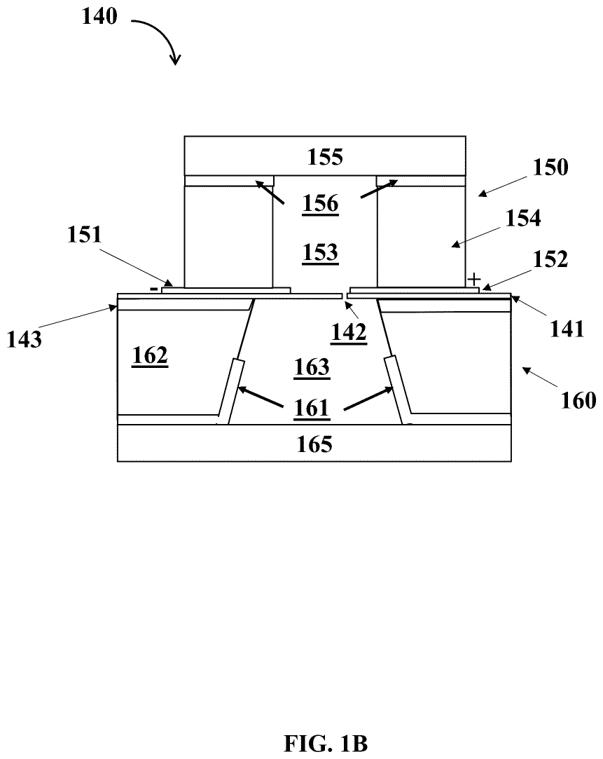

FIG. 1A depicts an example nanopore unit cell, as disclosed herein. The nanopore unit cell 100 can comprise a top cavity 110 and a bottom cavity 120. The top cavity 110 can comprise a top chamber 113 filled with a first dielectric, e.g., fluid 1. The bottom cavity can comprise a substrate 122, a bottom chamber 123 filled with a second dielectric, e.g., fluid 2. The nanopore unit cell 100 can comprise a dielectric membrane 101 that is supported by the substrate 122 and configured to separate the top cavity 110 and the bottom cavity 120. The dielectric membrane 101 can comprise a nanopore site 102. The nanopore unit cell can further comprise at least two electrodes, e.g., a positive electrode 112 and a negative electrode 111, adjacent to and associated with the nanopore site 102. In some cases, the nanopore unit cell can further comprise at least two, at least three, at least four, or more additional electrodes. The two electrodes 111 and 112 can comprise sharp pointed edges (or share edges, or sharp convex tips, used interchangeably herein). In some cases, the nanopore unit cell can comprise a top electrode 116 and a bottom electrode 121, a dielectric separator 114 that isolates electrodes 111 and 112 from electrode 116. In some cases, the nanopore unit cell 100 can further comprise a top lid 115 and a bottom lid 125. The top and bottom chambers can be enclosed with the top and bottom lids to create a tight seal to enable fluid flow in and out of the top and bottom chambers.

FIG. 1B depicts another example nanopore unit cell, as disclosed herein. The nanopore unit cell 140 can comprise a top cavity 150 and a bottom cavity 160. The top cavity 150 can comprise a top chamber 153 filled with a first dielectric. The bottom cavity can comprise a substrate 162, a bottom chamber 163 filled with a second dielectric. The nanopore unit cell 140 can comprise a dielectric membrane 141 that is supported by the substrate 162 and configured to separate the top cavity 150 and the bottom cavity 160. The dielectric membrane 141 can comprise a nanopore site 142. The nanopore unit cell can further comprise a second dielectric membrane 143. The nanopore unit cell can further comprise at least two electrodes, e.g., a positive electrode 152 and a negative electrode 151, adjacent to and associated with the nanopore site 142. In some cases, the nanopore unit cell can further comprise at least two, at least three, at least four, or more additional electrodes. The two electrodes 151 and 152 can comprise sharp pointed edges. In some cases, the nanopore unit cell can comprise a top electrode 156 and a bottom electrode 161, a dielectric separator 154 that isolates electrodes 151 and 152 from electrode 156. In some cases, the nanopore unit cell 140 can further comprise a top lid 155 and a bottom lid 165. The top and bottom chambers can be enclosed with the top and bottom lids to create a tight seal to enable fluid flow in and out of the top and bottom chambers.

In some embodiments, each nanopore unit cell can be used for processing a fluid sample or analyzing biomolecules independently.

In some embodiments, the electrodes associated with the nanopore site can be coated with a thin layer of a dielectric material. For example, if the electrodes associated with the nanopore site are built from aluminum, a thin layer of Al₂O₃ passivation layer can be coated on the surface of the electrodes associated with the nanopore site. In some embodiments, the electrodes associated with the nanopore site can be used as a tunneling current sensor to detect the translocating biomolecules. The thin layer of dielectric material on the electrodes associated with the nanopore site can be useful to measure the tunneling currents.

In some embodiments, the nanopore device can comprise a nanopore chip, comprising a plurality of nanopore unit cells as disclosed herein. Nanopore unit cells arranged and fabricated in an array to form a nanopore chip can increase the throughput of processing samples or analyzing biomolecules.

The nanopore chip can comprise a dielectric membrane comprising a plurality of nanopore sites, wherein each nanopore site is disposed between a top cavity comprising a first dielectric and a bottom cavity comprising a second dielectric, and associated with at least two electrodes, comprising a positive electrode and a negative electrode, adjacent to and associated with the nanopore site.

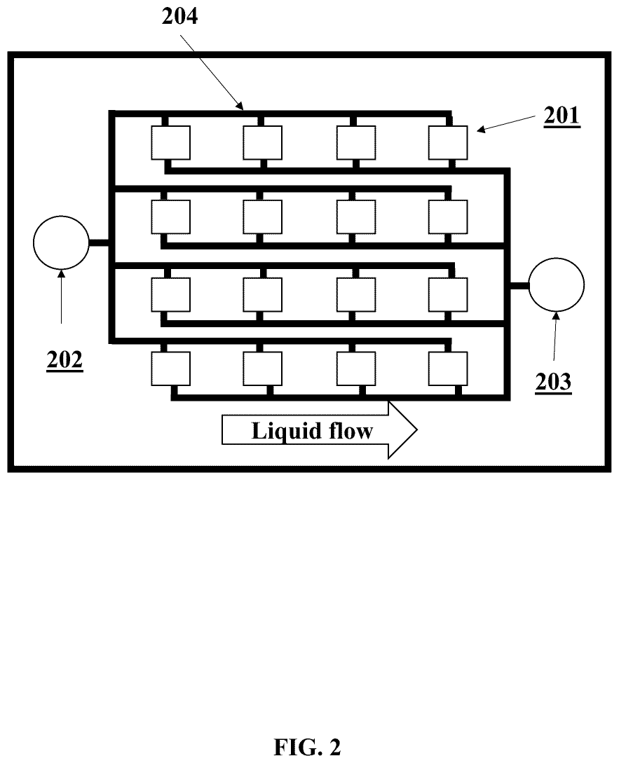

The nanopore chip can be fabricated on a substrate, e.g., silicon wafer, with any suitable micro/nano fabrication techniques. In some embodiments, a nanopore chip can be used for analyzing one sample. In some embodiments, a nanopore chip can be used for analyzing a plurality of samples. FIG. 2 shows a top view schematic of an example nanopore chip with a plurality of nanopore unit cells (e.g., nanopore unit cell block). The plurality of nanopore unit cells (e.g., 201) can be arranged in an array (e.g., an ordered array) and connected to an inlet reservoir 202 and an outlet reservoir 203 through microfluidic channels (e.g., 204). In some embodiments, the microfluidic channels can have a width from about 1 micrometer (μm) to about 10 μm, from about 1 μm to about 20 μm, from about 1 μm to about 30 μm, from about 1 μm to about 40 μm, from about 1 μm to about 50 μm, from about 1 μm to about 100 μm, from about 10 μm to about 20 μm, from about 10 μm to about 30 μm, from about 10 μm to about 40 μm, from about 10 μm to about 50 μm, from about 10 μm to about 100 μm, from about 20 μm to about 30 μm, from about 20 μm to about 40 μm, from about 20 μm to about 50 μm, from about 20 μm to about 100 μm, from about 30 μm to about 40 μm, from about 30 μm to about 50 μm, from about 30 μm to about 100 μm, from about 40 μm to about 50 μm, from about 40 μm to about 100 μm, or from about 50 μm to about 100 μm. In some embodiments, each nanopore unit of the plurality of unit cells can be connected to the inlet reservoir to receive a portion of the sample from the inlet reservoir. In some embodiments, each nanopore unit of the plurality of unit cells can be connected to the outlet reservoir to direct the portion of the sample from the nanopore unit cell to the outlet reservoir. Alternatively, a first nanopore unit cell of the plurality of unit cells can be connected to the inlet reservoir to receive a portion of the sample from the inlet reservoir and a second nanopore unit cell of the plurality of unit cells can be connected to the first nanopore unit cell, not the inlet reservoir, to receive the portion of the sample from the first nanopore unit cell. In some embodiments, the fluid samples (e.g., samples comprising biomolecules) to be analyzed can be introduced into the inlet reservoir 202 and driven to the plurality of nanopore unit cell via pressure driven, capillary, electrowetting mechanism, or electro osmotic flow. After the samples are analyzed in the nanopore unit cell, the samples can be driven to the outlet reservoir 203. The electrodes within each nanopore unit cell can be routed for independent access similar to a DRAM chip.

In some embodiments. a nanopore chip can comprise from 10 to 100, from 10 to 500, from 10 to 1000, from 10 to 10000, from 10 to 100000, from 10 to 1 million, from 10 to 500, from 10 to 1000, from 10 to 10000, from 10 to 100000, from 10 to 1 million, from 100 to 500, from 100 to 1000, from 100 to 10000, from 100 to 100000, from 100 to 1 million, from 500 to 1000, from 500 to 10000, from 500 to 100000, from 500 to 1 million, from 1000 to 10000, from 1000 to 100000, from 1000 to 1 million, from 10000 to 100000, from 10000 to 1 million, or from 100000 to 1 million nanopore unit cells.

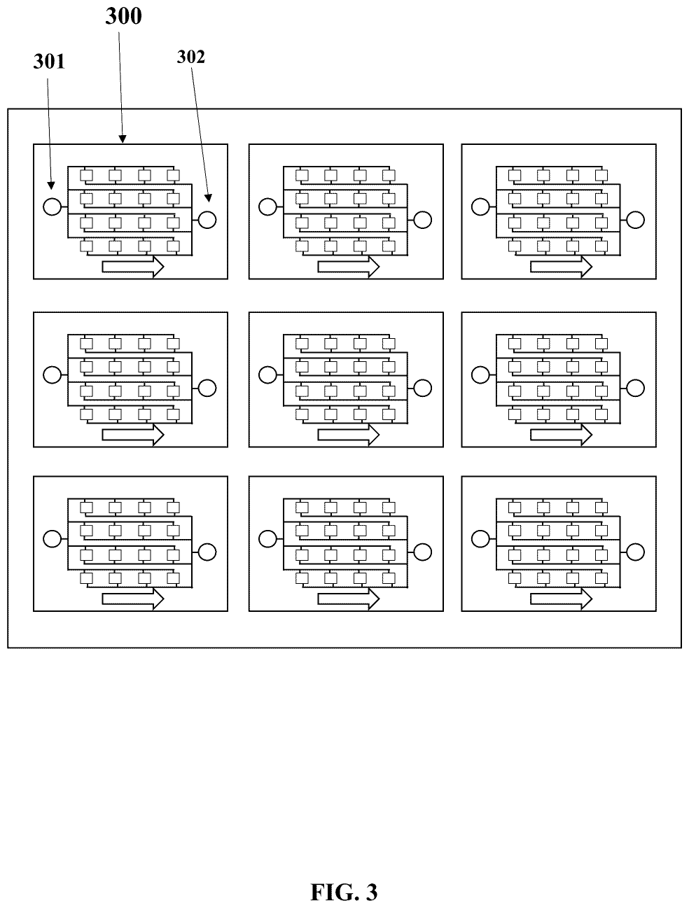

In some embodiments. the nanopore unit cells can be organized in an array, e.g., nanopore array. In some embodiments, the nanopore array can be used for multiplex sample analysis in one analysis run. FIG. 3 shows an example nanopore array. The nanopore array can comprise a plurality of nanopore unit cell blocks (e.g., nanopore unit cell block 300), wherein each of the nanopore unit cell blocks can comprise a plurality of nanopore unit cells, an inlet reservoir 301 and an outlet reservoir 302. The nanopore array can be fabricated by the massively parallel micro/nano fabrication process. The samples can be loaded to the inlet reservoirs of different nanopore unit cells by a microfluidic network or through robotics automation.

### Systems for Processing Samples or Analyzing Biomolecules

In some embodiments, the present disclosure provides a system for processing a sample, characterizing, analyzing, or sequencing a biomolecule, comprising a nanopore device as disclosed herein.

In some embodiments, the system provided herein can be utilized in biomolecule screening (e.g., pathogen detection), diagnostics, DNA/RNA/protein fingerprinting and purification, drug discovery and development, NGS sample preparation, nanoparticle synthesis (e.g., fluid 1 & 2 can be nanoparticle source materials), food and water quality testing and purification, and/or general filtration applications using electric fields.

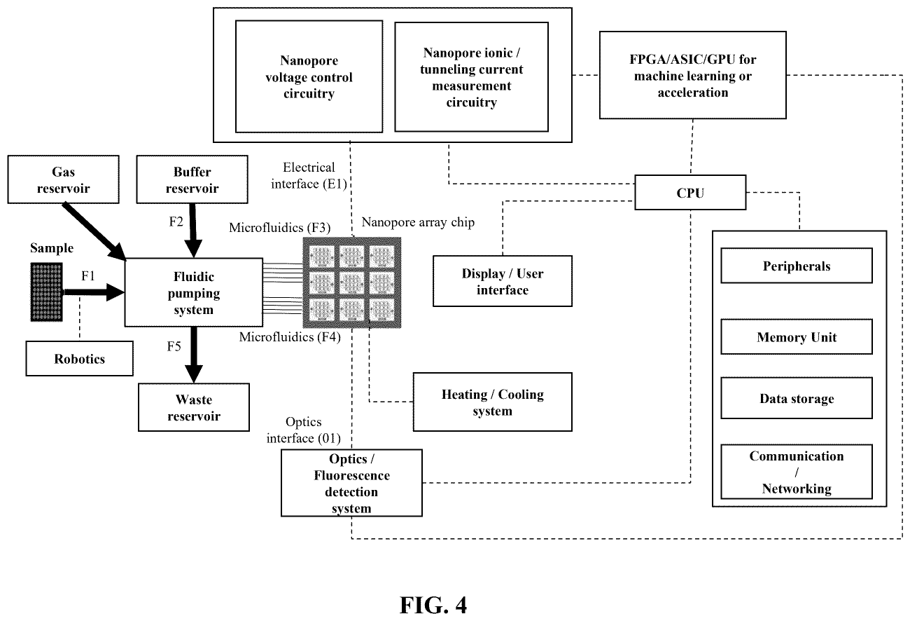

FIG. 4 shows an example system for sample processing or biomolecule analysis. The system can comprise a nanopore device (e.g., nanopore array chip).

In some embodiments, the system can comprise a gas reservoir, buffer reservoir, and/or waste reservoir. The gas(es) may fill the top and bottom chambers of the nanopore unit cells. In some embodiments, the system can comprise interfaces for microfluidics to direct fluid(s) (e.g., gas, sample, buffer, etc.) to the inlets of the nanopore unit cell blocks and direct the fluid(s) from the nanopore unit cell blocks to a waste reservoir. The sample(s) to be analyzed can be loaded to the system in various formats, for example: 1) 96 well plate; 2) 384 well plate; 3) 8 well strip tubes; or 4) single well centrifuge tubes.

In some embodiments, the system can comprise a fluidic pumping system configured to pump the samples into and out of the nanopore array chip, pumping gas or buffer from gas or buffer reservoirs to and from the chip, and pumping the waste from the chip to a waste reservoir, In some embodiments, the nanopore array chip may comprise one or more microfluidic channels interfacing with the fluidic pumping system.

In some embodiments, the system can comprise a robotics system to load the samples and coordinate the interfacing between a fluidic pumping system and the sample(s).

In some embodiments, the system can further comprise voltage control and current measurement circuits, CPU, memory unit, and data storage. In some embodiments, the system can comprise sample(s) and loading system, optical system, heating/cooling system, peripherals, communication/networking, and display systems.

In some embodiments, the system can comprise a nanopore voltage control circuitry configured to provide voltage to the top electrode and bottom electrode (e.g., 116 and 121 of FIG. 1A and 156 and 161 of FIG. 1B) for biomolecule translocation and/or ionic current measurement. In some embodiments, the nanopore voltage control circuitry can be configured to provide voltage to the positive and negative electrodes (e.g., 111 and 112 of FIG. 1A and 151 and 152 of FIG. 1B) for tunneling current measurement.

In some embodiments, the system can comprise a nanopore ionic/tunneling current measurement circuitry configured to measure electrical signals, e.g., the currents, when the sample or biomolecules translocate through the nanopore. In some embodiments, the nanopore ionic/tunneling current measurement circuitry can report the measured electrical signals to an accelerator (e.g., FPGA/ASIC/GPU) to run machine-learning models to convert the electrical signals to biomolecule information. Alternatively or in addition to, the nanopore ionic/tunneling current measurement circuitry can send the measured electrical signals to a CPU for analysis at the CPU.

The CPU can act as a central controller for data produced by the system and interface with a display, user interface, other components (e.g., memory, data storage, networking, etc.), and/or peripherals (I/O). The system can be controlled via a touch screen user interface or a computer using a wired interface (e.g., USB, Ethernet) or a wireless interface. In some embodiments, the wireless interface can comprise a mobile device. A software can be used for analysis of the biomolecules and producing the size distribution information. The software can interface with automation frameworks such as LIMS for automation of workflows.

In some embodiments, the system can comprise an optics/fluorescence detection system configured to detect the biomolecules via optical or fluorescence methods. In some embodiments, the optics/fluorescence detection system can interface with the CPU/accelerators for signal processing.

In some embodiments, the nanopore of the nanopore array chip can be generated in situ. When the nanopore array chip is loaded into the system, the fluidic pumping system pumps the dielectric compositions (e.g., gases or liquids) to the top and the bottom chambers of the nanopore unit cell, then the nanopore voltage control circuitry and the current measurement circuitry drives the chip under the influence of the CPU to create the nanopore.

In some embodiments, biomolecule sequencing can be performed with this system with the signals obtained from the ionic/tunneling current sensors. In some embodiments, the nanopore array chip can have a membrane that is a few atoms thick. In some embodiments, the membrane can be a 2-dimensional material (e.g., graphene, MoS₂) or ultra-thin membranes. This can enable the measurement of current variations at the single base/amino acid level. In some embodiments, the ionic/tunneling currents (e.g., raw current signal data) measured can be stored locally or in a cloud. The ionic/tunneling currents measured can be fed to the detection circuit and the accelerators for base calling. The CPU can store the base calls in a file or upload it to the cloud.

In some embodiments, the system disclosed herein can be capable of maintaining the nanopores without clogs. For example, if a biomolecule or other debris clogs the nanopore, a corona discharge and/or are discharge can be used to unclog the nanopores and regenerate the nanopore.

### Methods of Manufacturing Nanopore Devices

In some embodiments, the present disclosure provides a method of manufacturing a nanopore device for processing a fluid sample, the method comprising: (a) providing a device, the device comprising: a substrate; a top cavity; a bottom cavity; a first dielectric membrane; and a first set and a second set of electrodes; and (b) applying a first electric field between the first and second sets of electrodes to generate a corona discharge near an edge of the first and second sets of electrodes; wherein the corona discharge forms one or more nanopores in the first dielectric membrane. The corona discharge can result in a high concentration of charged particles near the electrodes, thereby creating the nanopore at the corona discharge location.

In some embodiments, (a) comprises: (A) depositing the first dielectric membrane on the substrate; (B) forming the bottom cavity in the substrate underneath the first dielectric membrane, wherein the bottom cavity comprises a dielectric (e.g., the second dielectric disclosed in the present disclosure); (C) depositing the first set and the second set of electrodes on top of the first dielectric membrane; and (D) forming the top cavity on top of the first dielectric membrane and on top of the first and second sets of electrodes, wherein the top cavity comprises an additional dielectric (e.g., the first dielectric disclosed in the present disclosure).

In some embodiments, the depositing in (C) comprises depositing the first set of electrodes proximate to the dielectric, the additional dielectric, and the first dielectric membrane. In some embodiments, the depositing in (D) comprises depositing the second set of electrodes proximate to the first dielectric, the second dielectric, and the first dielectric membrane.

In some embodiments, the method can comprise generating the first electric field by applying a pulsed or continuous AC or DC voltage signal between the first and second sets electrodes. In some embodiments, the first electric field can comprise a level of at least about 1 kV/m, at least about 10 kV/m, at least about 50 kV/m, 100 kV/m, at least about 1 megavolt/meter (MV/m), at least about 10 MV/m, at least about 100 MV/m, or greater.

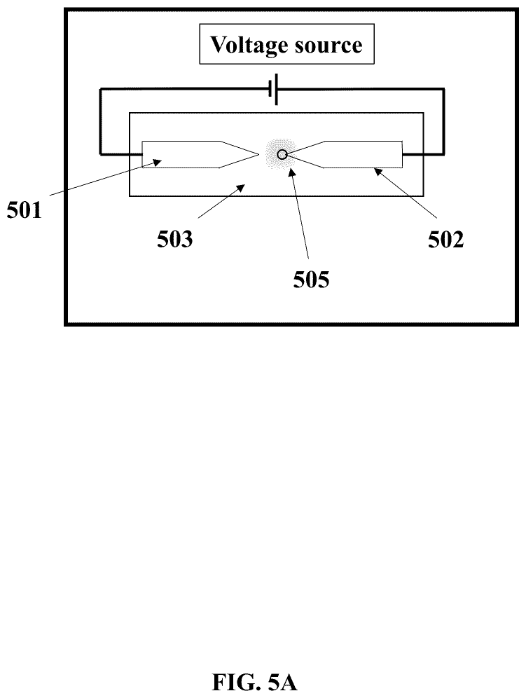

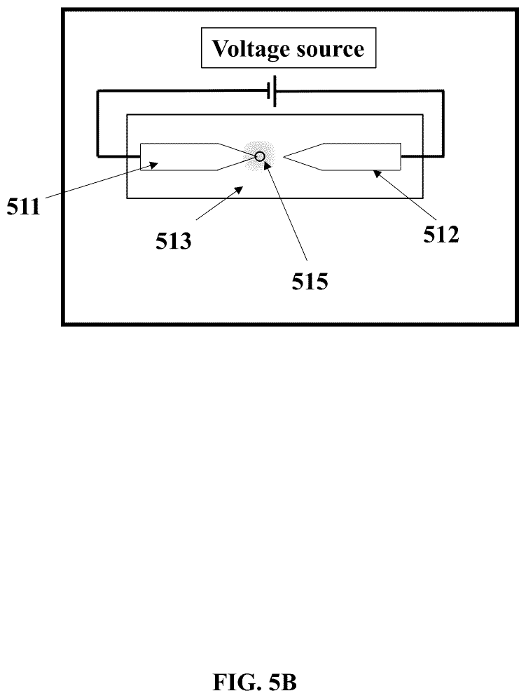

FIGS. 5A and 5B show example setups for generating corona discharge. A continuous or pulsed DC or AC voltage is applied between two electrodes 501 and 502, and 511 and 512, e.g., conducting metal electrodes, with sharp edges and placed close to each other, surrounded by a dielectric 503 and 513, e.g., liquid or gas. A high concentration of electric field exists at the sharp edges of the electrodes due to a field gradient. When the electric field is high enough (e.g., higher than 30 kv/m for air), electrons escaping the electrodes can ionize atoms of the dielectric 503 surrounding the electrodes. The ionization can create an electron-ion pair with an ionic current, resulting in breakdown of the dielectric material. When these ions and electrons travel towards electrodes of opposite polarity, recombination of the charged particles result in a glow or corona discharge. In some cases, the corona discharge can be formed at the sharp edge of the positive electrode, e.g., a positive corona discharge 505, as shown in FIG. 5A. In some cases, the corona discharge can be formed at the sharp edge of the negative electrode, e.g., a negative corona discharge 515, as shown in FIG. 5B. This can result in emission of photons and an increase in the temperature. The amount of charged particles, photons (light), the radius of the corona discharge, and the temperature increase may depend on the applied voltage and pressure. The location where the corona is formed can have a higher concentration of charged particles (e.g., electrons, ions).

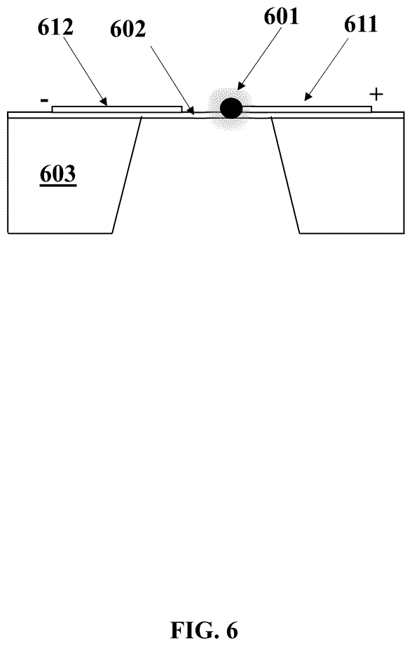

FIG. 6 shows an example setup for forming a nanopore. A dielectric membrane 602 is supported by a substrate 603 and separates a top cavity and a bottom cavity. In both cavities, the dielectrics can be air. When the two electrodes (e.g., 611 and 612) are placed on the dielectric membrane 602 supported by the substrate 603, with an applied voltage, the corona discharge 601 formed can be used to etch or melt the dielectric membrane 602 at the location of the corona discharge, thereby creating a nanopore at the location of the corona discharge. In some cases, an additional dielectric membrane can be disposed between the dielectric membrane 602 and the substrate 603. In some embodiments, the nanopore can have a diameter from about 0.1 nanometers (nm) to about 1 nm, from about 0.1 nm to about 5 nm, from about 0.1 nm to about 10 nm, from about 0.1 nm to about 50 nm, from about 0.1 nm to about 100 nm, from about 0.1 nm to about 500 nm, from about 0.1 nm to about 1 micrometer (μm), from about 0.1 nm to about 10 μm, from about 1 nm to about 5 nm, from about 1 nm to about 10 nm, from about 1 nm to about 50 nm, from about 1 nm to about 100 nm, from about 1 nm to about 500 nm, from about 1 nm to about 1 μm, from about 1 nm to about 10 μm, from about 5 nm to about 10 nm, from about 5 nm to about 50 nm, from about 5 nm to about 100 nm, from about 5 nm to about 500 nm, from about 5 nm to about 1 μm, from about 5 nm to about 10 μm, from about 10 nm to about 50 nm, from about 10 nm to about 100 nm, from about 10 nm to about 500 nm, from about 10 nm to about 1 μm, from about 10 nm to about 10 μm, from about 50 nm to about 100 nm, from about 50 nm to about 500 nm, from about 50 nm to about 1 μm, from about 50 nm to about 10 μm, from about 100 nm to about 500 nm, from about 100 nm to about 1 μm, from about 100 nm to about 10 μm, from about 500 nm to about 1 μm, from about 500 nm to about 10 μm, or from about 1 μm to about 10 μm.

In some cases, the chambers need not be structural features, but rather areas that are positions relative to one another and separated by the dielectric membrane.

In some cases, any dielectric can be selected (including various gases or liquids) or a low conductive dielectric solid can be selected as the first dielectric or the second dielectric.

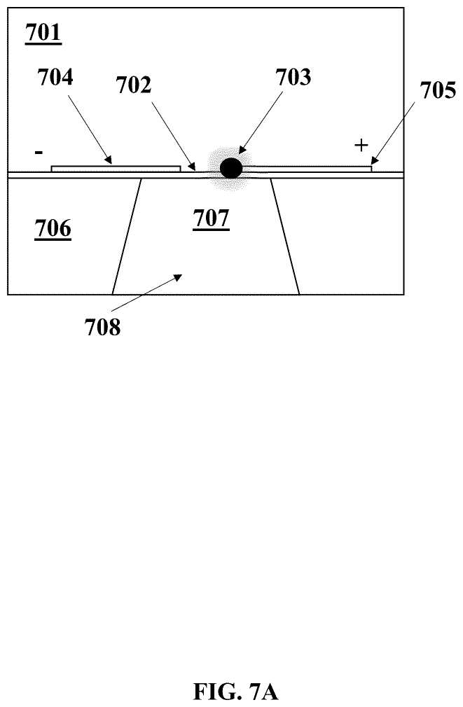

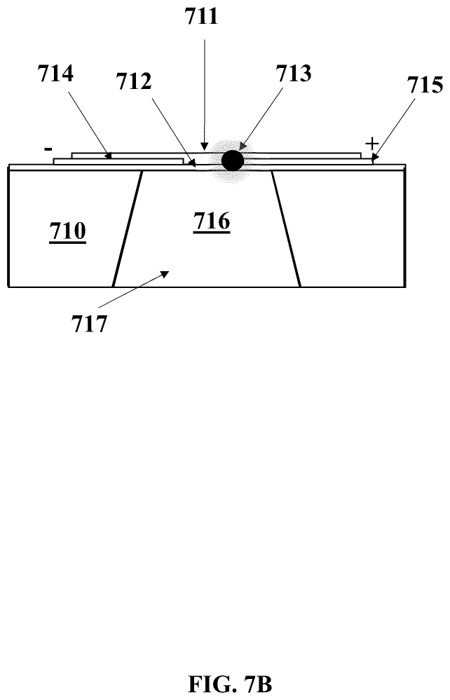

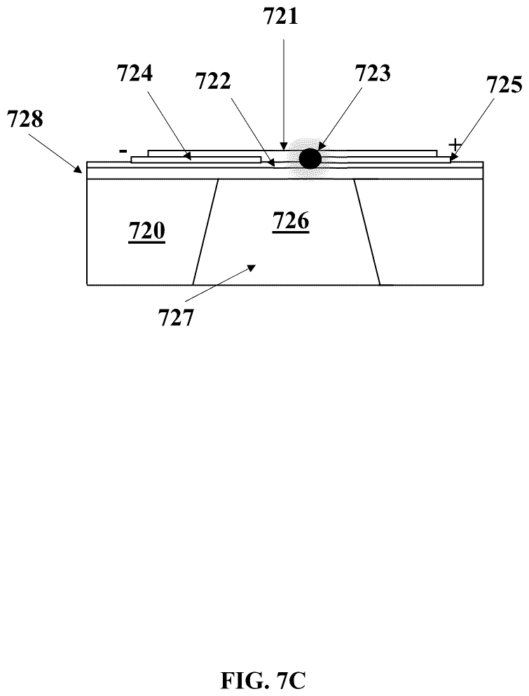

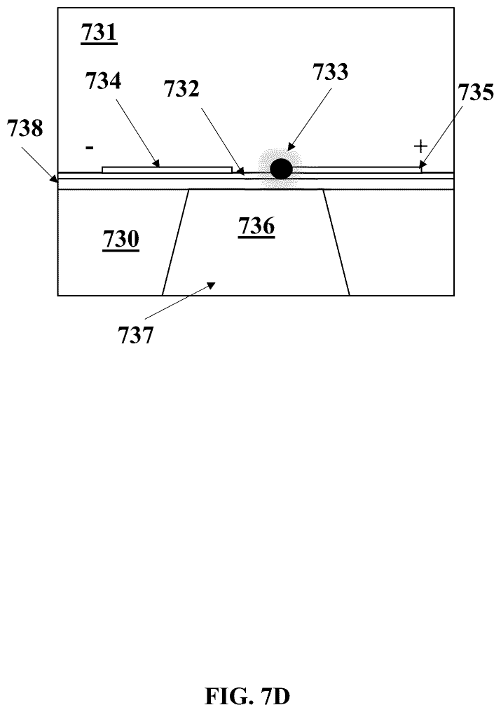

FIG. 7A shows another example setup for forming a nanopore. A dielectric membrane 702 is supported by a substrate 706 and separates a top cavity and a bottom cavity. The top chamber of the top cavity is filled with a first dielectric, e.g., fluid 701, and the bottom chamber of the bottom cavity is filled with a second dielectric, e.g., fluid 707. Fluids 701 and 707 can be any combination of gas or liquid. In some embodiments, the top and bottom chambers can be filled with the same fluid. In some embodiments, the top and bottom chambers can be filled with different fluids. The corona discharge 703 formed can be used to etch or melt the dielectric membrane at the location of the corona discharge, thereby creating a nanopore at the location of the corona discharge.

FIG. 7B shows another example setup for forming a nanopore. A dielectric membrane 712 is supported by a substrate 710. Two electrodes 714 and 715 are placed adjacent to the dielectric membrane 712. A dielectric solid material 711 surrounds the two electrodes. The bottom chamber 717 of the bottom cavity can comprise a dielectric 716, which can be any combination of gas or liquid. The dielectric solid material 711 may comprise a predefined concentration of dopant ions. In some embodiments, a corona discharge 713 can be formed. In some embodiments, the corona discharge may not be visible or present, which is called partial discharge and can create erosion of the dielectric membrane. The corona discharge 713 formed can be used to etch or melt the dielectric membrane material at the location of the corona discharge, thereby creating a nanopore at the location of the corona discharge.

In some embodiments, a second dielectric membrane can be used. In some embodiments, the second dielectric membrane can be disposed between the dielectric membrane and the substrate. In some cases, the dielectric membrane and the additional dielectric membrane can have a substantially same size. In some cases, the dielectric membrane and the additional dielectric membrane can have different sizes. In some embodiments, the second dielectric membrane can be disposed between the first dielectric membrane and the substrate during the formation of the nanopore, and can be removed after the nanopore formation.

FIG. 7C shows another example setup for forming a nanopore. A dielectric membrane 722 (e.g., a first dielectric membrane) is supported by an additional dielectric membrane 728 (e.g., a second dielectric membrane) and the additional dielectric membrane 728 is support by a substrate 720). Two electrodes 724 and 725 are disposed adjacent to the dielectric membrane 722 and coated with dielectric solid material 721. The bottom chamber 727 of the bottom cavity can comprise a dielectric 726, which can be any combination of gas or liquid. The dielectric solid material 721 may comprise a predefined concentration of dopant ions. In some embodiments, a corona discharge 723 can be formed. In some embodiments, the corona discharge may not be visible or present, which is called partial discharge and can create erosion of the dielectric membrane. The corona discharge 725 formed can be used to etch or melt the dielectric membrane material at the location of the corona discharge, thereby creating a nanopore at the location of the corona discharge. The additional dielectric membrane 728 may be removed, e.g., via chemical etching, after the nanopore formation. In some cases, a nanopore can be formed through both the dielectric membrane 722 and the additional dielectric membrane 728.

FIG. 7D shows another example setup for forming a nanopore. A dielectric membrane 732 (e.g., a first dielectric membrane) is supported by an additional dielectric membrane 738 (e.g., a second dielectric membrane) and the additional dielectric membrane 738 is support by a substrate 730. The dielectric membrane 732 and the additional dielectric membrane 738 separate the top cavity and the bottom cavity. Two electrodes 734 and 735 can be disposed adjacent to the dielectric membrane 732. The top chamber of the top cavity can comprise a dielectric 731 (e.g., a first dielectric, fluid, or gas). The bottom chamber 737 of the bottom cavity can comprise a dielectric 736 (e.g., a second dielectric, fluid, or gas). The dielectrics 731 and 736 can be any combination of gas or liquid. The corona discharge 733 formed can be used to remove a portion of the dielectric membrane material at the location of the corona discharge, thereby creating a nanopore at the location of the corona discharge. The additional dielectric membrane 738 may be removed above the dielectric 736, e.g., via chemical etching. after the nanopore formation. In some embodiments, the additional dielectric membrane 738 located above dielectric 736 can be etched away after the nanopore formation. In some cases, a nanopore can be formed through both the dielectric membrane 732 and the additional dielectric membrane 738.

In some embodiments, the device can further comprise a second dielectric membrane. In some embodiments, (a) can further comprise depositing the second dielectric membrane between the first dielectric membrane and the substrate or the second dielectric.

In some embodiments, the device can further comprise a top layer and a bottom layer. In some embodiments, (a) can further comprise forming the top layer to a top of the first dielectric and forming the bottom layer to a bottom of the second dielectric.

The corona discharge is a non-thermal process and may produce an increase in temperature of the device or near a nanopore site. In some embodiments, the temperature increase can be from about 0.1° C., to about 10° C. In some embodiments, the intensity of charge and temperature at the corona discharge location may not be high enough to etch thick membranes to create a nanopore. To solve this issue, a second electric field can be configured to generate an electrical are discharge, which can be applied in addition to the corona discharge, to produce a local electric field to assist the nanopore formation. In the corona discharge-assisted dielectric are discharge nanopore creation method, the corona discharge location can act as a concentrator of the electric field lines. Thus, it can enable localizing the nanopore creation.

In some embodiments, the present disclosure provides a nanopore creation method with the corona discharge-assisted dielectric are discharge provided herein.

In some embodiments, the present disclosure provides a method of manufacturing a nanopore device for processing a fluid sample. the method comprising: (i) providing a device, the device comprising: a substrate; a top cavity; a bottom cavity; a first dielectric membrane; a first set and a second set of electrodes; and a third set and a fourth set of electrodes; (ii) applying a first electric field between the first and second sets of electrodes to generate a corona discharge near an edge of the first and second sets of electrodes; and (iii) applying a second electric field between the third and fourth sets of electrodes to generate an electrical are discharge, wherein a combination of the corona discharge and the electrical are discharge form one or more nanopores in the first dielectric membrane.

In some embodiments, the corona discharge generated in (ii) can result in a high concentration of charged particles near the electrodes. In some embodiments, (iii) can comprise applying the second electric field between the third and fourth sets of electrodes, until the second electric field exceeds a threshold such that dielectric strength of fluid in a top or bottom cavity breaks down and starts conducting current, resulting in are discharge and breakdown of the dielectric membrane.

In some embodiments, (i) can comprise: (1) depositing the first dielectric membrane on the substrate; (2) forming the bottom cavity in the substrate underneath the first dielectric membrane, wherein the bottom cavity comprises a dielectric (e.g., the second dielectric); (3) depositing the first set and the second set of electrodes on top of the first dielectric membrane; (4) forming the top cavity on top of the first dielectric membrane and on top of the first and second sets of electrodes, wherein the top cavity comprises an additional dielectric (e.g., the first dielectric); (5) depositing the third set of electrodes on a top side of the top cavity; and (6) depositing the fourth set of electrodes on a bottom side of the bottom cavity.

In some embodiments, the depositing in (3) can comprise depositing the first set and the second set of electrodes proximate to the first dielectric, and the first dielectric membrane.

In some embodiments, the depositing in (5) can comprise depositing the third set of electrodes proximate to the first dielectric. In some embodiments, the depositing in (6) can comprise depositing the fourth set of electrodes proximate to the second dielectric.

In some embodiments, the method can comprise generating the first electric field by applying a pulsed or continuous AC or DC voltage signal between the first and second sets electrodes. In some embodiments, the first electric field can comprise a level of at least about 1 kV/m, at least about 10 kV/m, at least about 50 kV/m, at least about 100 kV/m, at least about 1 MV/m, at least about 10) MV/m, at least about 100 MV/m, or greater.

In some embodiments, the method can comprise generating the second electric field by applying a pulsed or continuous AC or DC voltage signal between the third and fourth sets electrodes. In some embodiments, the second electric field can comprise a level of at least about 1 kV/m, at least about 10 kV/m, at least about 50 kV/m, at least about 100 kV/m, at least about 1 MV/m, at least about 10 MV/m, at least about 100 MV/m, at least about 1000 MV/m, at least about 5000 MV/m, or greater.

In some embodiments, the device can further comprise a second dielectric membrane. In some embodiments, (i) can further comprise depositing the second dielectric membrane between the first dielectric membrane and the substrate or the second dielectric.

In some embodiments, the device can further comprise a top layer and a bottom layer. In some embodiments, (i) can further comprise forming the top layer to a top of the first dielectric and forming the bottom layer to a bottom of the second dielectric.

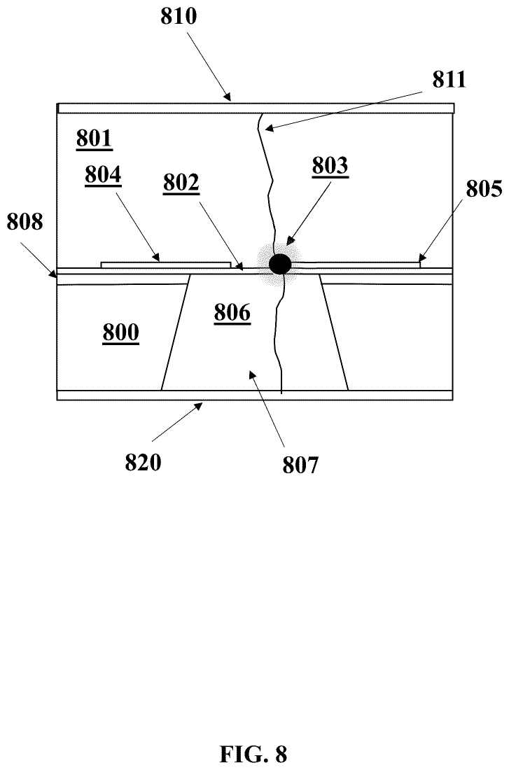

FIG. 8 shows an example setup for corona discharge-assisted dielectric are discharge nanopore creation. A dielectric membrane 802 is supported by an additional dielectric membrane 808 and the additional dielectric membrane 808 is support by a substrate 800. The dielectric membrane 802 and the additional dielectric membrane 808 can separate the top cavity and the bottom cavity. In some cases, the dielectric membrane and the additional dielectric membrane can have a substantially same size. In some cases, the dielectric membrane and the additional dielectric membrane can have different sizes. The additional dielectric membrane 808 can be disposed between the dielectric membrane 802 and the substrate 800. In some embodiments, the additional dielectric membrane may not be present, and the dielectric membrane 802 can be supported by the substrate and can separate the top cavity and the bottom cavity. Two electrodes 804 and 805 are disposed adjacent to the dielectric membrane 802. The top chamber of the top cavity can comprise a first dielectric 801. The bottom chamber 807 of the bottom cavity can comprise a second dielectric 806. The dielectrics 801 and 806 can be any combination of gas or liquid. As depicted in FIG. 8, the corona discharge 803 is created by applying a pulsed or continuous DC or AC field between the electrodes 804 and 805 with sharp features (e.g., sharp edges). This may result in a high concentration of charged particles near the electrodes 804 and 805. When an external electric field (e.g., a second electric field) is applied with the external electrodes, e.g., external electrodes 810) and 820, the external electric field (811 shows example field lines) can concentrate at the corona discharge location, thus producing a much higher concentration of charges at the corona discharge location. This can lead to a nanopore creation at the corona discharge location.

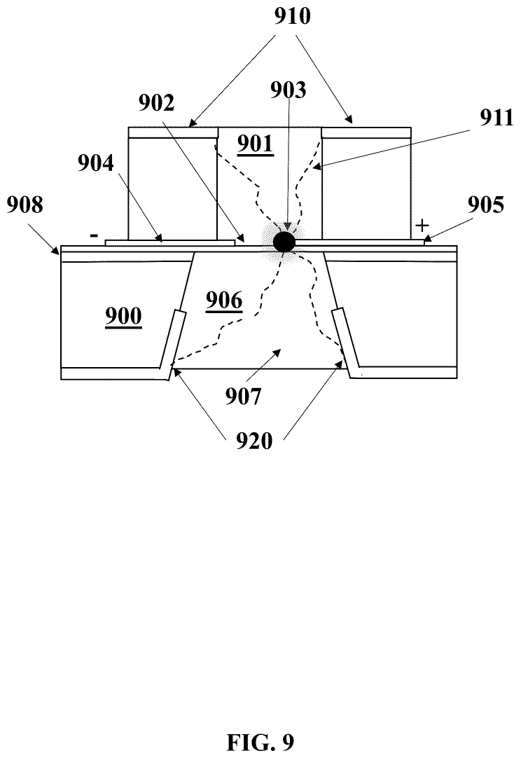

In some embodiments, the external electrodes can be connected to the fluids in the top and bottom chambers (as shown in FIG. 8). In some embodiments, the external electrodes can be embedded in the top chamber and the bottom chamber. FIG. 9 shows another example setup for corona discharge-assisted dielectric are discharge nanopore creation. A dielectric membrane 902 (e.g., a first dielectric membrane) is supported by an additional dielectric membrane 908 (e.g., a second dielectric membrane) and the additional dielectric membrane 908 is support by a substrate 900. In some cases, the dielectric membrane and the additional dielectric membrane can have a substantially same size. In some cases, the dielectric membrane and the additional dielectric membrane can have different sizes. The additional dielectric membrane 908 can be disposed between the dielectric membrane 902 and the substrate 900. The dielectric membrane 902 and the additional dielectric membrane 908 can separate the top cavity and the bottom cavity. In some embodiments, the additional dielectric membrane may not be present, and the dielectric membrane 902 can be supported by the substrate and can separate the top cavity and the bottom cavity. Two electrodes 904 and 905 are disposed adjacent to the membrane 902. The top chamber of the top cavity can comprise a first dielectric 901. The bottom chamber 907 of the bottom cavity can comprise a second dielectric 906. The dielectrics 901 and 906 can be any combination of gas or liquid. The setup can further comprise an electrode 910 embedded within the top cavity and an electrode 920 embedded within the bottom cavity. As depicted in FIG. 9, the corona discharge 903 is created by applying a pulsed or continuous DC or AC field (e.g., a first electric field) between the electrodes 904 and 905 with sharp features (e.g., sharp edges). This can result in a high concentration of charged particles near the electrodes. When an external electric field (e.g., a second electric field) is applied with the external electrodes, e.g., external embedded electrodes 910 and 920, the electric field (911 shows example field lines) can concentrate at the corona discharge location, thus producing a much higher concentration of charges at the corona discharge location. This can lead to a nanopore creation at the corona discharge location.

When the external electrodes are embedded in the chambers, each of the nanopore creating blocks can be considered as a unit cell and mass produced in wafer scale to produce millions of nanopores in a controlled fashion. The fluids in the top and bottom chambers can be enclosed to form a microfluidic channel or network to transport fluids to the nanopore location. In some embodiments, the microfluidic channel or network can be used in transporting biomolecules for analysis to the nanopore unit cells.

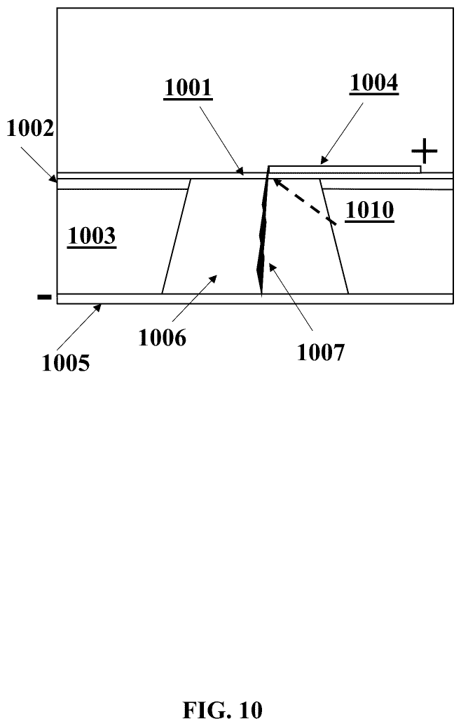

FIG. 10 shows an example setup for are discharge nanopore creation. A dielectric membrane 1001 (e.g., a first dielectric membrane) is supported by an additional dielectric membrane 1002 (e.g., a second dielectric membrane) and the additional dielectric membrane 1002 is support by a substrate 1003. In some cases, the dielectric membrane and the additional dielectric membrane can have a substantially same size. In some cases, the dielectric membrane and the additional dielectric membrane can have different sizes. The additional dielectric membrane 1002 can be disposed between the dielectric membrane 1001 and the substrate 1003. The membrane 1001 and the additional membrane 1002 can separate the top cavity and the bottom cavity. In some embodiments, the additional dielectric membrane may not be present, and the dielectric membrane 1001 can be supported by the substrate and can separate the top cavity and the bottom cavity. The top chamber of the top cavity can comprise a first fluid and the bottom chamber 1006 of the bottom cavity can comprise a second fluid. A first electrode 1004 with a sharp edge is placed on top of the membrane 1001 and a second electrode 1005 is placed at the bottom of the substrate 1003 and an electric field is applied between the first electrode and the second electrode. When the electric field exceeds a threshold, the dielectric strength of the fluid in the bottom chamber 1006 breaks down and the fluid starts conducting current, generating an are discharge 1007. This results in the breakdown of the dielectric membrane(s), creating a nanopore 1010 in the membrane and the optional additional membrane when it is present.

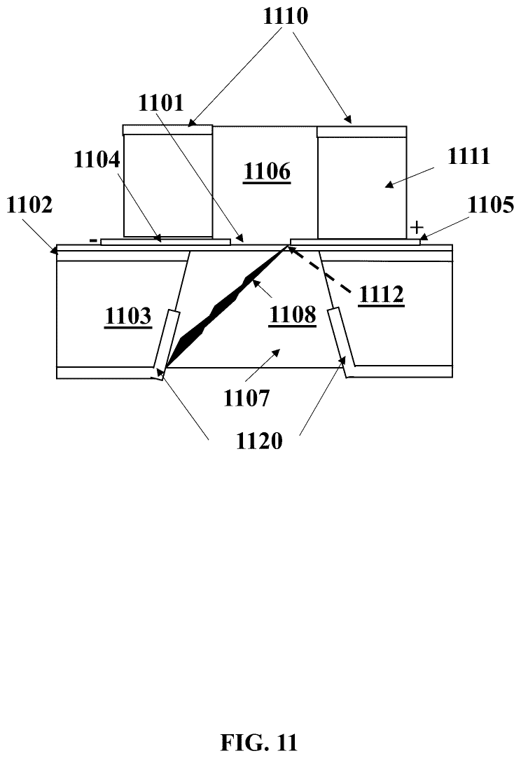

FIG. 11 shows another example setup for are discharge nanopore creation. A dielectric membrane 1101 (e.g., a first dielectric membrane) is supported by an additional dielectric membrane 1102 (e.g., a second dielectric membrane) and the additional dielectric membrane 1102 is support by a substrate 1103. In some cases, the dielectric membrane and the additional dielectric membrane can have a substantially same size. In some cases, the dielectric membrane and the additional dielectric membrane can have different sizes. The additional dielectric membrane 1102 can be disposed between the dielectric membrane 1101 and the substrate 1103. The membrane 1101 and the additional membrane 1102 can separate the top cavity and the bottom cavity. In some embodiments, the additional dielectric membrane may not be present, and the dielectric membrane 1101 can be supported by the substrate and can separate the top cavity and the bottom cavity. The top chamber 1106 of the top cavity a can comprise a first fluid and the bottom chamber 1107 of the bottom cavity can comprise a second fluid. Two electrodes 1104 and 1105 comprising sharp edges are placed on top of the membrane 1101. External electrode 1110 is embedded within the top cavity and a dielectric material 1111 is disposed between and separates the external electrode 1110 and electrodes 1104 and 1105. Another external electrode 1120 is embedded within the bottom cavity. When an electric field is applied between the electrode 1105 and the external electrode 1120 and the electric field exceeds a threshold. the dielectric strength of the fluid in the bottom chamber 1107 breaks down and the fluid starts conducting current, generating an are discharge 1108. This results in the breakdown of the dielectric membrane(s), creating a nanopore 1112 in the dielectric membrane and the optional additional dielectric membrane when it is present.

Unlike the corona discharge-based method, the are discharge can produce intense heat (e.g., >1000° C.) to melt the dielectric membrane in its path. In some embodiments, the are discharge nanopore creation method can be used to create nanopores for thicker membranes, e.g., membranes with about 1 nm to 50 nm thickness.

In some embodiments, any method of manufacturing the nanopore device disclosed herein can further comprise depositing a fifth and a sixth electrodes on top of the first dielectric membrane. In some embodiments, the fifth and sixth electrodes can be configured to generate a dielectrophoretic (DEP) force with the first and second sets of electrodes.

In some embodiments, any method of manufacturing the nanopore device disclosed herein can further comprise: (I) generating a plurality of the nanopore devices; (II) electrically connecting at least one nanopore device of the plurality of nanopore devices to at least one other nanopore device of the plurality of nanopore devices to form a plurality of nanopore cells; and (III) electrically connecting at least one nanopore cell of the plurality of nanopore cells to at least one other nanopore cell of the plurality of nanopore cells to form a plurality of nanopore arrays. The plurality of nanopore arrays can be configured to perform high-throughput processing of the fluid sample.

### Methods of Using Nanopore Devices

In some embodiments, the present disclosure provides a method for processing a fluid sample using one or more nanopores, the method comprising: (a) providing a device, the device comprising: a substrate; a top cavity; a bottom cavity; a first dielectric membrane; a first set and a second set of electrodes configured to generate a corona discharge; and a third set and a fourth set of electrodes configured to generate an electrical are discharge, wherein the one or more nanopores are formed in the first dielectric membrane using a combination of the corona discharge and the electrical are discharge; (b) flowing the fluid sample through the one or more nanopores; and (c) processing the fluid sample as the fluid sample flows through the one or more nanopores.

In some embodiments, the method can comprise using the first, second, third, or fourth sets of electrodes to form the one or more nanopores. In some embodiments, the one or more nanopores can be formed prior to contacting the first dielectric membrane with the fluid sample.

In some embodiments, the method can further comprise using the first and second sets of electrodes to detect one or more events associated with the processing of the fluid sample.

In some embodiments, the fluid sample can comprise deoxyribonucleic acid (DNA), ribonucleic acid (RNA), proteins, lipids, or carbohydrates suspended in a gas, a water, an electrolyte fluid, or a porous solid (e.g., a gel). In some embodiments, the one or more events can comprise events associated with analyzing biomolecules, measuring biomolecules, purifying biomolecules, concentrating biomolecules, or sequencing biomolecules. In some embodiments, (b) can comprise flowing the fluid sample through the one or more nanopores via capillary, pressure driven, electro osmotic flow, or electrowetting mechanism.

In some embodiments, processing a fluid sample can comprise analyzing a biomolecule, synthesis, purification, processing, and/or sequencing a DNA, RNA, or protein.

In some embodiments, the method can comprise using the first and second sets of electrodes to measure a tunnel current. In some embodiments, the method can comprise using the third and fourth sets of electrodes to measure an ionic current.

In some embodiments, the device can further comprise a fifth electrode and a sixth electrode configured to generate a dielectrophoretic (DEP) force with the first and second sets of electrodes. In some embodiments, the method can further comprise using the DEP force to concentrate biomolecules contained in the fluid sample.

In some embodiments, the method can further comprise using one or more sensor to measure an ionic current and/or tunneling current.

### Computer Systems

The present disclosure provides computer systems that are programmed to implement one or more methods of the present disclosure. Computer systems of the present disclosure may be used to regulate various operations of nanopore sequencing and/or analysis, such as detecting one or more signals when a sample is within the nanopore (e.g., a protein nanopore or a solid state nanopore).

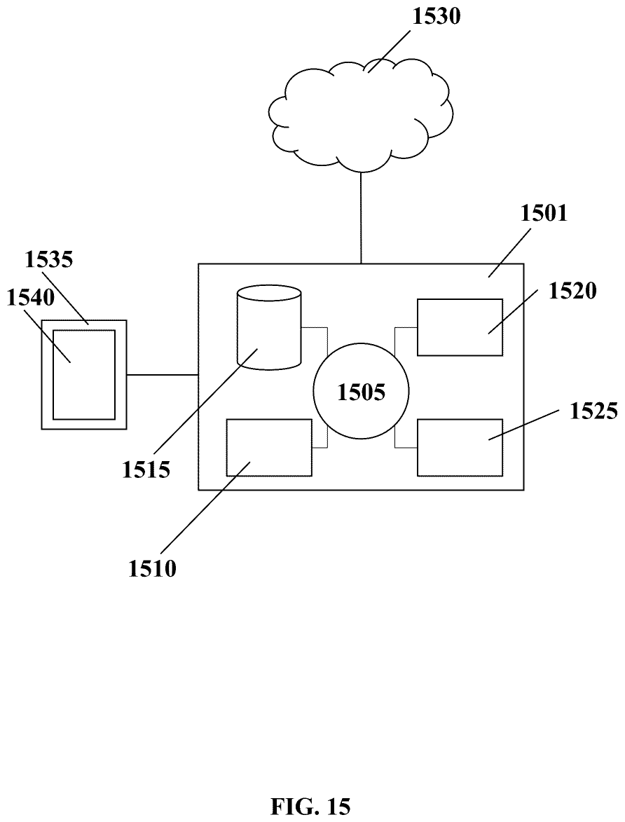

FIG. 15 shows a computer system 1501 that is programmed or otherwise configured to communicate with and regulate various aspects of sequencing and/or analysis of the present disclosure. The computer system 1501 can communicate with, for example, one or more circuitry coupled to or comprising a nanopore (or a membrane comprising the nanopore), and one or more devices (e.g., machines) used to prepare, treat, or keep one or more reaction mixtures for sequencing and/or analysis. The computer system 1501 may also communicate with one or more controllers or processors of the present disclosure. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk or solid state drive), communication interface 1520) (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the

Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC), field-programmable gate array (FPGA), digital signal processor (DSP), or graphics processing unit (GPU).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine-readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, (i) progress of the reaction mixture, (ii) progress of sequencing, and (iii) sequencing information obtained from sequencing. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, determine sequence readout of one or more target sites upon nanopore sequencing.

### EXAMPLES

The following examples are provided to further illustrate some embodiments of the present disclosure but are not intended to limit the scope of the disclosure; it will be understood by their exemplary nature that other procedures, methodologies, or techniques may alternatively be used.

### Example 1

### Exemplary Biomolecule Analysis System

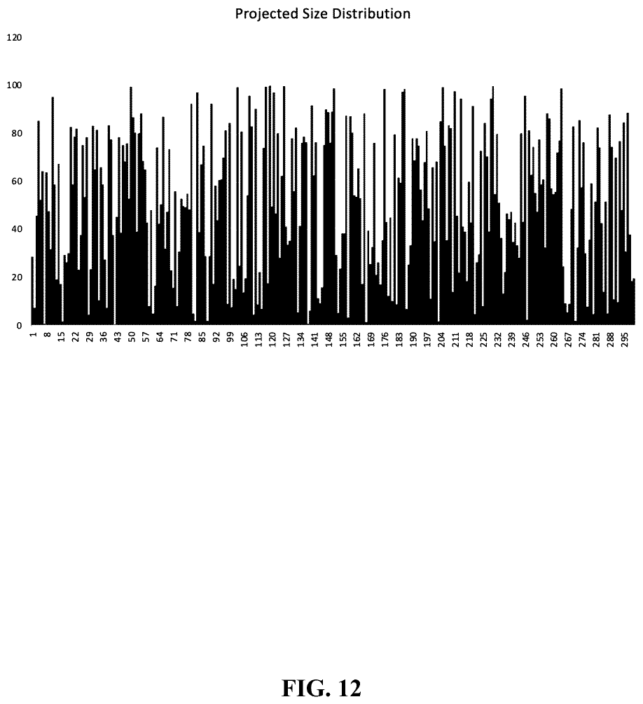

The biomolecule analysis system disclosed herein enables single base/amino acid resolution high-throughput size distribution analysis of biomolecules. In other words, this system enables obtaining a high resolution size distribution chart, as depicted in FIG. 12. The accurate measurement of biomolecule sizes (e.g., lengths of DNA expressed in kilobases) is useful in determining the efficacy of assays such as polymerase chain reaction (PCR).

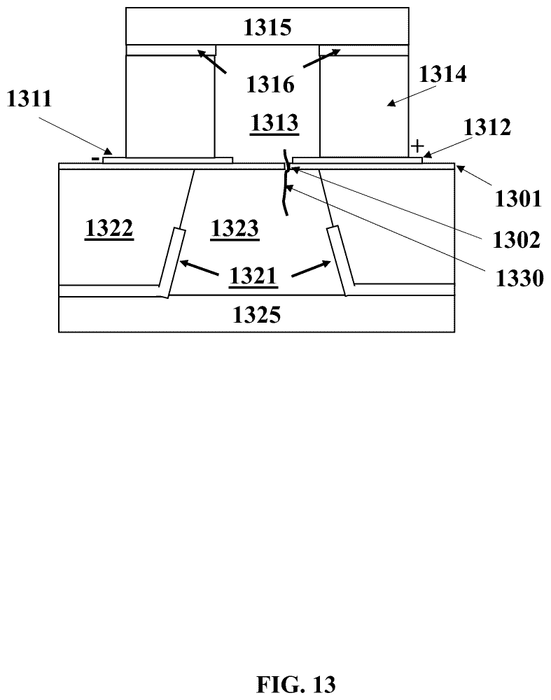

FIG. 13 depicts a biomolecule translocating from a top chamber to a bottom chamber of a nanopore unit cell. The nanopore unit cell can comprise a top cavity and a bottom cavity. The top cavity can comprise a top chamber 1313 filled with a first dielectric. The bottom cavity can comprise a substrate 1322, a bottom chamber 1323 filled with a second dielectric. The nanopore unit cell can comprise a dielectric membrane 1301 that is supported by the substrate 1322 and configured to separate the top cavity and the bottom cavity. The dielectric membrane 1301 can comprise a nanopore site 1302. The nanopore unit cell can further comprise two electrodes, e.g., a positive electrode 1312 and a negative electrode 1311, adjacent to and associated with the nanopore site 1302. The two electrodes 1311 and 1312 can comprise sharp pointed edge. In some cases, the nanopore unit cell can comprise a top electrode 1316 and a bottom electrode 1321, a dielectric separator 1314 that isolates electrodes 1311 and 1312 from electrode 1316. In some cases, the nanopore unit cell can further comprise a top lid 1315 and a bottom lid 1325. The top and bottom chambers can be enclosed with the top and bottom lids to create a tight seal to enable fluid flow in and out of the top and bottom chambers.

The biomolecule translocation 1330 can be driven by an applied electric field between electrodes 1316 and 1321. The biomolecule translocation can result in ionic current change between electrodes 1316 and 1321, and tunneling current change between electrodes 1311 and 1312.

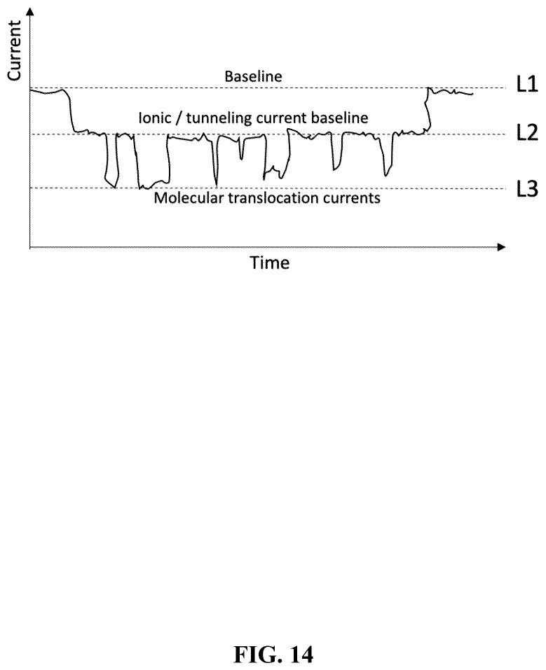

FIG. 14 shows an exemplary measurement of current vs time in the nanopore. The baseline current (L1) is the current level when there is no ion flow in the nanopore, the ionic/tunneling current baseline (L2) is the current when an ion blocks the nanopore or the tunneling sensor, and the molecular translocation current (L3) is the measured current by the ionic/tunneling sensor when a biomolecule translocates through the nanopore. When there is an ion blockade at the nanopore or tunneling sensor, the current decreases from the baseline L1 to L2. When a biomolecule translocates through the nanopore, the current decreases to L3. The length of the biomolecule can be determined by the magnitude of the current decrease and the duration of the decrease.

### Example 2

### Exemplary Biomolecule Analysis with a Nanopore Unit Cell

With reference to FIG. 13, the bottom chamber 1323 is filled with an ionic liquid one. A sample comprising biomolecules in ionic liquid two is introduced into the top chamber 1313. An electric field applied between electrodes 1316 and 1321 can translocate the biomolecules through the nanopore from the top chamber (cis) to the bottom chamber (trans).

In the absence of biomolecules, the passage of ions through the nanopore creates perturbations in the measured ionic current between electrodes 1316 and 1321. These perturbations are due to the blockade of the ionic current flow. When a biomolecule passes through the nanopore, the ionic current blockade is much higher, thus resulting in a much lower ionic current. The ionic current can be measured with an electrode immersed in the liquid in the top and bottom chambers. The electrodes are embedded in the nanopore unit cell which enables independent control of individual nanopore unit cell. Further, this enables parallel analysis of large numbers of biomolecules with large numbers (e.g., millions) of nanopores.

The electrodes 1311 and 1312 that were used to fabricate the nanopore with the corona or are discharge can be used as tunneling current sensors. The electrodes 1311 and 1312 can be passivated with an insulating oxide layer. When a high electric field is applied between the electrodes 1311 and 1312, a small amount of current flows between the two electrodes through quantum mechanical tunneling. When a biomolecule passes through the nanopore it can affect the charge density in the gap between the electrodes 1311 and 1312. This can result in a change in the tunneling current between the electrodes thus providing important information about the biomolecule passing through the nanopore.

The ionic current and tunneling current and changes thereof can be used in detecting, analyzing and manipulating biomolecules in the nanopore chip.

While preferred embodiments of the present disclosure have been shown and described herein, such embodiments are provided by way of example only. Numerous variations, changes, and substitutions can occur without departing from the disclosure. It can be understood that various alternatives to the embodiments of the present disclosure may be employed in practicing the present disclosure. It is intended that the following claims define the scope of the present disclosure and that methods and structures within the scope of these claims and their equivalents be covered thereby.

1-76. (canceled)

77. A nanopore device for processing a fluid sample, comprising:
a substrate;
a top cavity, wherein the top cavity comprises a first dielectric;
a bottom cavity, wherein the bottom cavity comprises a second dielectric;
a first dielectric membrane supported by the substrate and separating the top cavity and the bottom cavity;
a first set and a second set of electrodes located proximate to the first dielectric membrane,
wherein the first set and the second set of electrodes are configured to apply a first electric field to generate a corona discharge thereby forming one or more nanopores in the first dielectric membrane.

78. The device of claim 77, further comprising a third set and a fourth set of electrodes configured to apply a second electric field, wherein the third set is located proximate to the first dielectric and the fourth set is located proximate to the second dielectric.

79. The device of claim 78, wherein the third and fourth sets of electrodes are configured to apply the second electric field to generate an electrical are discharge to thereby form the one or more nanopores in the first dielectric membrane.

80. The device of claim 79, wherein the combination of the corona discharge and the electrical are discharge is configured to form the one or more nanopores in the first dielectric membrane.

81. The device of claim 79, wherein the third and fourth sets of electrodes are configured to generate the electrical are discharge through the first dielectric, the second dielectric, or the first dielectric membrane.

82. The device of claim 79, wherein the third and fourth sets of electrodes are configured to apply the second electric field before contacting the device with the fluid sample or before generating the corona discharge.

83. The device of claim 79, wherein the second electric field is configured to generate the electrical are discharge by exceeding a dielectric breakdown of the first dielectric, the second dielectric, or the first dielectric membrane.

84. The device of claim 79, wherein the corona discharge is configured to concentrate the second electric field near the corona discharge to improve a location accuracy of the one or more nanopores by at least about 5% compared to without use of the corona discharge.

85. The device of claim 79, wherein the third set of electrodes is configured to electrically couple to the first dielectric, and wherein the fourth set of electrodes is configured to electrically couple to the second dielectric.

86. The device of claim 79, wherein the third and fourth sets of electrodes are configured to be integrated into the device, and wherein the third and fourth sets of electrodes are fabricated from a material comprising one or more combinations of gold, chromium, aluminum, platinum, iridium, or titanium.

87. The device of claim 86, further comprising a second electric field generator, wherein the second electric field generator is configured to apply the second electric field sufficient to generate the electrical are discharge by applying a pulsed or continuous alternating current (AC) or direct current (DC) voltage signal between a pair of the third and fourth sets of electrodes.

88. The device of claim 87, wherein the second electric field generator is configured to generate the second electric field with a strength of at least about 1 kV/m.

89. The device of claim 77, wherein the one or more nanopores comprise a diameter ranging from about 0.1 nanometers (nm) to about 10 micrometers (μm).

90. The device of claim 78, further comprising one or more sensors configured to measure a tunneling current between a pair of the first and second sets of electrodes or an ionic current between a pair of the third and fourth sets of electrodes.

91. The device of claim 77, further comprising a second dielectric membrane, wherein the second dielectric membrane forms a layer between the first dielectric membrane and the substrate or the second dielectric.

92. The device of claim 91, wherein the second dielectric membrane is configured to support the first dielectric membrane during the forming of the one or more nanopores and to be removed after the forming of the one or more nanopores.

93. The device of claim 77, wherein the device is configured to reform the one or more nanopores in the first dielectric membrane.

94. The device of claim 77, further comprising a top layer coupled to the first dielectric and a bottom layer coupled to the second dielectric, wherein the top and bottom layers are configured to contain or seal the fluid sample or guide a flow of the fluid sample through the device.

95. The device of claim 77, further comprising a fifth electrode and a sixth electrode configured to generate a dielectrophoretic (DEP) force with the first and second sets of electrodes.

96. The device of claim 95, wherein the DEP force is used to concentrate biomolecules contained in the fluid sample.
